SEQUENCE LISTING (6 sequences)

SEQ ID NO: 1 - protein, 25 aa, homo sapiens
YGRKKRRQRRRGKPPAPPMRNTSTM

SEQ ID NO: 2 - protein, 13 aa, homo sapiens
KPPAPPMRNTSTM

SEQ ID NO: 3 - protein, 12 aa, human immunodeficiency virus
YGRKKRRQRRRG

SEQ ID NO: 4 - protein, 18 aa, homo sapiens
PPPVIAPRPEHTKSVYTR

SEQ ID NO: 5 - protein, 30 aa, homo sapiens
YGRKKRRQRRRGPPPVIAPRPEHTKSVYTR

SEQ ID NO: 6 - protein, 30 aa, homo sapiens
YGRKKRRQRRRGPPPVIAPAAEHAKSVYTR

(19) United States
(12) Patent Application Publication
(10) Pub. No.: US 2009/0022712 A1
(43) Pub. Date: Jan. 22, 2009

(54) Compositions and Methods for Inhibiting Leukocyte Function

(75) Inventors: Joerg Reutershan, Ammerbuch (DE); Martin A. Schwartz, Earlysville, VA (US); Klaus F. Ley, La Jolla, CA (US); Rebecca A. Stockton, Del Mar, CA (US)

Correspondence Address: UNIVERSITY OF VIRGINIA PATENT FOUNDATION, 250 WEST MAIN STREET, SUITE 300, CHARLOTTESVILLE, VA, 22902, US

(73) Assignee: UNIVERSITY OF VIRGINIA PATENT FOUNDATION, Charlottesville, VA (US)

(21) Appl. No.: 12/161,915

(22) PCT Filed: Jan. 24, 2007
(86) PCT No.: PCT/US07/02077

§ 371 Date: Jul. 23, 2008

Related U.S. Application Data
(60) Provisional application No. 60/761,471, filed on Jan. 24, 2006.

(51) Int. Cl. A61K 39/395; C12N 5/06; C12N 15/85; A61K 31/7088; A61K 38/10; A61P 31/00; A61P 25/00; A61P 13/12; A61P 17/00; A61P 11/00; A61P 1/00; A61P 35/00; A61P 29/00; A61K 31/7105; C12N 5/08; A61K 38/17

(52) U.S. Cl. 424/130.1; 435/375; 435/455; 435/372; 435/29; 514/12; 514/14; 514/13; 435/15; 514/44

(57) ABSTRACT

The present invention provides compositions and methods for regulating leukocyte migration and function. The present invention also provides compositions and methods for preventing and inhibiting lung injury and damage associated with neutrophil infiltration of the lung.

### CROSS REFERENCE TO RELATED APPLICATIONS

This application is entitled to priority pursuant to 35 U.S.C. § 119(e) to U.S. provisional patent application No. 60/761,471, filed on Jan. 24, 2006, the entirety of which is incorporated by reference herein.

### STATEMENT REGARDING FEDERALLY SPONSORED RESEARCH OR DEVELOPMENT

This invention was made with United States Government support under Grant Nos. GM47214 and HL73361, awarded by the National Institutes of Health. The United States Government has certain rights in the invention.

### BACKGROUND

Acute inflammatory diseases are characterized by rapid recruitment of polymorphonuclear neutrophils (PMNs). In acute bacterial infections, this PMN recruitment can be protective, but in many diseases such as ventilator-induced lung injury, influenza infection, acute lung injury (ALI) or acute respiratory distress syndrome (ARDS), PMN recruitment is inappropriate and leads to tissue damage, sometimes resulting in the death of the patient.

Various strategies have been developed to curb neutrophil recruitment, including blockade of leukocyte adhesion molecules by antibodies, peptides, or small molecule. Some of these strategies have resulted in useful therapeutic agents that are on the market today, such as an LFA-1 antibody produced by Genentech that is effective in psoriasis. Another approach to blocking PMN recruitment is to block G-protein coupled chemoattractant receptors, for example the CXC chemokine receptor 2.

The PMN cytoskeleton consists of F-actin and hundreds of proteins regulating its polymerization in time and space. Migration requires continuous and rapid remodeling of the cytoskeleton through pathways that are currently being discovered. These pathways are attractive targets for pharmacological manipulation of neutrophil migration, because migration is central to neutrophil function. Neutrophil and other inflammatory cell (monocyte, macrophage, dendritic cell, T cell, NK cell, NKT cell) migration is 10-100 times faster than migration of non-inflammatory cells, suggesting that selective blockage can be achieved without disturbing other body functions.

In experimental studies, depletion of PMNs has been demonstrated to curb lung damage (1). Clinical observations suggest that lung function in ARDS patients negatively correlates with neutrophil counts in the blood (2). Although there is compelling evidence that PMN infiltration is functionally significant in ALI/ARDS, there are currently no clinically validated strategies to modulate neutrophil recruitment. This deficiency is most likely due to our incomplete understanding of the molecular mechanisms underlying PMN migration into the lung. Mortality in ALI/ARDS is still high, and specific therapies beyond mechanical ventilation and other supportive approaches are not available (3).

The molecular requirements for PMN trafficking into inflamed lungs differ fundamentally from those in other tissues (4) and are strongly dependent on the model of lung injury. Selectins and their ligands, essential to initiate the contact between PMNs and the endothelium in many vascular beds, are not required to mediate PMN adhesion in the lung where PMN activation might be sufficient to allow PMN lodging in small capillaries of the pulmonary microcirculation (5). However, neutrophil transmigration into the lung interstitium and alveolar airspace requires molecules on both PMNs and endothelium, including adhesion molecules and chemokine receptors, as shown in studies with genetically modified mice and antibody blocking strategies (6), (7). After adhesion to the endothelium is established, transmigration is initiated by stimulus-induced cytoskeletal reorganization of PMNs and endothelial cells. Actin is polymerized at the leading edge of neutrophils, forming a lamellipod that mediates directional movement toward the chemoattractant. These processes require small GTPases such as Rac and Cdc42, both of which have been demonstrated to play a key role in the directed movement and migration of PMNs to sites of inflammation (8, 9).

The small GTPases Rac and Cdc42 are central organizers of the neutrophil cytoskeleton. These GTPases are activated in neutrophils and other leukocytes in response to bacterial products, inflammatory chemokines and cytokines, and cell contact-dependent signaling events during infection and injury. p21-activated kinases (PAK) 1, 2, and 3 constitute a family of serine/threonine kinases activated by Rac and Cdc42 (10). PAK regulates both actin polymerization and myosinphosphorylation to control motility. Additionally, PAK is a component of the NADPH oxidase complex that produces reactive oxygen that is a significant factor in tissue damage.

The catalytic domain is highly conserved between PAK isoforms and across species. Both PAK1 (11, 12) and PAK (13, 14) expression have been demonstrated in neutrophils, where they are involved in chemotaxis. Because the inhibitory peptide does not distinguish between PAK isoforms (20), the term is PAK to indicate any of the three isoforms throughout the text.

Under resting conditions, PAK forms a homodimer in which catalytic activity is blocked by binding an inhibitory domain in the N-terminus to the opposing catalytic subunit (15). Activation of PAK involves phosphorylation of the thr423 and ser141 residues, dissociation of the dimer and release of the catalytic domains (6). PAK binds to SH3-containing adaptor proteins, including Nck and PAK-associated guanine nucleotide exchange factor (PIX) which mediate translocation of PAK to targets at the cell membrane and cell-cell junctions (17, 18), ultimately resulting in cytoskeletal remodeling and cell contractility (19). The sequence within PAK which binds to PIXα and PIXβ is PPPVIAPRPEHTKSVYTR (SEQ ID NO:4) (Manser et al., Mol. Cell. 1998 1:2:183-92). Interfering with PAK interaction with Nck blocks endothelial migration and angiogenesis (20). In endothelial cells, cytokine-induced PAK phosphorylation and translocation to cell-cell junctions has been demonstrated to increase monolayer permeability in vitro (21). Additionally, PAK translocation to integrin-mediated focal complexes and focal adhesions has been reported in epithelial cells (22) and fibroblasts (23) where it mediates the formation of actin microspikes and induces loss of adhesions and stress fibers, suggesting a role for cell spreading and transmigration (24). In neutrophils, PAK is activated by chemoattractants such as fMLP (25, 26) and implicated in the directional movement of PMNs towards a chemotactic gradient in vitro (27).

There is a long felt need in the art for new compositions and methods for regulating leukocyte migration. The present invention satisfies these needs.

### BRIEF SUMMARY OF INVENTION

The present disclosure identifies PAK as a central mediator of neutrophil-dependent lung injury. PAK blockade inhibits chemotaxis, actin polymerization, and adhesion-induced oxidative burst in mouse and human neutrophils. The present invention therefore encompasses targeting PAK pathways in leukocyte associated diseases and disorders, including lung diseases and disorders, and inflammation associated with increased leukocyte activity or infiltration.

A tat-linked peptide that blocks PAK 1,2 and 3 blocks neutrophil migration in vitro and neutrophil recruitment in models of lung inflammation in mice in vivo. Application of the peptide has-no apparent adverse side effects. The blockade of neutrophil migration into the lung is almost 100%, making this peptide much more effective than other known anti-inflammatory treatments. Without wishing to be bound by any particular theory, it is hypothesized that peptides inhibiting PAK function inhibit leukocyte adhesion and migration and are therefore useful for the development of anti-inflammatory drugs. PAK 1, 2 and 3 are therefore encompassed herein as targets for anti-inflammatory drug development. The human orthologues of mouse PAKs are known. (gi|427947691NP_{—}002567 for PAK1, gi|32483399NP_{—}002568 for PAK2 and gi|47117818|O075914 for PAK3) and highly homologous to mouse PAK (88% identical amino acid sequence for PAK1). According to one aspect of the invention, one or more of the PAK family members may be inhibited by peptides or other means, or their interaction with other molecules in the cell may be altered, blocked, enhanced, or modified in any way.

The present invention encompasses, inter alia, all applications in which any PAK family member is targeted into any leukocyte, including neutrophils, eosinophils, basophils, T lymphocytes, B lymphocytes, natural killer (NK) cells, NKT cells, monocytes, macrophages, dendritic cells, and the derivatives and motile precursors of these leukocytes, by any means, including peptides, small molecules, dominant negatives, antisense or small interfering RNA, aptamers, antibodies, natural substances, or other means. In one aspect, the invention provides compositions and methods for inhibiting leukocyte migration. In one aspect, the leukocyte is a neutrophil. In one aspect, the composition comprises the tat-PAK peptide. In one aspect, the tat-PAK peptide and other molecules of the invention inhibit PMN recruitment to the lung and into bronchoalveolar lavage fluid. The methods and compositions encompassed by the present invention are therefore useful for treating acute or chronic lung injury or diseases or conditions associated with an influx of neutrophils.

In one embodiment, the tat-PAK peptide and other molecules encompassed by the invention inhibit leukocyte function. In one aspect, the peptides of the invention inhibit neutrophil migration by inhibiting a PAK pathway in endothelial cells (see FIG. 4). In one aspect, the inhibited function is one which is induced by growth factors, chemokines, other peptides, or endogenous or exogenous inflammatory mediators. In one aspect, the chemokine is MIP-2 (CXCL2). The present invention further provides drugs and other molecules, their precursors, derivatives which regulate PAK synthesis, levels, or activity. The present invention further provides methods for altering phosphorylation of the PAK family of molecules, as well as for other modifications of the PAK family molecules.

The present invention is applicable to all leukocytes, which includes neutrophils, eosinophils, basophils, T lymphocytes, B lymphocytes, natural killer (NK) cells, NKT cells, monocytes, macrophages, dendritic cells, and the derivatives and motile precursors of these leukocytes. To this end, the present application discloses the strong inhibitory effects of a specific PAK-inhibiting peptide in a model of acute lung inflammation.

The present invention encompasses peptides, and biologically active modifications, fragments, derivatives, homologs, and analogs thereof, which compete for interaction sites to disrupt PAK and PEK regulatory pathways, including signal transduction pathways, associated with leukocyte function. Such compounds are referred to as inhibitory or competitive compounds. In one aspect, the inhibitory compounds of the invention inhibit protein complex formation or interaction.

The present invention provides compositions and methods for diagnostic, therapeutic, preventative, cosmetic, lifestyle and other applications, whenever PAK family members in leukocytes are targeted. Many of the most likely applications will be in inflammatory diseases, some examples of which are listed below. This invention may also be useful in diseases that are not primarily inflammatory but have an inflammatory component, for example, in cancer where inhibiting inflammation may decrease growth or metastasis.

In one embodiment, the present invention provides a method of treating all forms of acute lung injury, including, but not limited to, ARDS, ventilator lung, sepsis-induced lung failure, certain forms of pneumonia, including influenza-induced pneumonia.

In another embodiment, the present invention provides a method of treating various forms of acute or chronic inflammation, including but not limited to, arthritis (rheumatoid and other forms), asthma, multiple sclerosis and other inflammatory diseases of the central nervous system, neuritis and other inflammatory diseases of the peripheral nervous system, atopic diseases, inflammatory bowel diseases, inflammatory diseases of the skin or mucosal membranes, hepatitis, glomerulonephritis and other inflammatory diseases of the kidney, sepsis including septic shock, cancers or other tumors with inflammatory components, cancers in which inhibition of cell migration would be beneficial, and any other inflammatory or other diseases in which inhibition of cell migration would be beneficial.

In one embodiment, the present invention provides a method of administering a peptide or other compound of the invention to a subject in need thereof. In accordance with one embodiment, a composition is provided comprising a PAK inhibitor and a pharmaceutically acceptable carrier. In one embodiment, the composition is formulated for intravenous delivery. The PAK inhibitor may be, for example, an agent which binds to, or blocks, either or both of the kinase domain of PAK and the p21 (e.g., Cdc42 or Rac1) binding domain of PAK, or the autophosphorylation sites of PAK. In one embodiment, the PAK inhibitor is a short peptide that contains the sequence from PAK that exerts dominant negative activity (Kiosses et al, 2002, Circ. Res. 90:697). This peptide (YGRKKRRQRRRGKPPAPPMRNTSTM; SEQ ID NO: 1) consists of the sequence KPPAPPMRNTSTM (SEQ ID NO:2) from the first proline-rich domain of PAK, fused to the polybasic sequence YGRKKRRQRRRG (SEQ ID NO:3) from the HIV TAT protein (Schwarze et al, 1999, Science 285:1569) which promotes entry into cells. The TAT sequence may also be used with other useful sequences. The peptide (SEQ ID NO: 1) inhibits PAK function similarly to full length dominant negative constructs. The peptide does not block PAK kinase activity per se, but instead displaces PAK from sites of action including cell-cell junctions, which is sufficient to prevent its effects on cellular contractility, migration, and permeability.

The sequence within PAK which binds to PDCβ and PIXβ is PPPVIAPRPEHTKSVYTR (SEQ ID NO:4). The present invention encompasses adding the TAT sequence to SEQ ID NO:4 for use in preventing or inhibiting the interaction of PAK with PIX.

The tat sequence of the human immunodeficiency virus (HIV) is commonly used to help peptides enter intact cells. The tat sequence does not carry any of the HIV pathogenicity and is widely used. Alternative sequences exist. Without wishing to be bound by any particular theory, it is hypothesized herein that such other sequences would be equally effective in promoting uptake of the peptide by PMNs and other inflammatory cells.

The present disclosure also encompasses other PAK regulators for use in the present invention. Assays useful for identifying additional PAK regulators have been described herein as well as in U.S. Pat. No. 6,248,549, in U.S. Patent Publication 20040138133, published Jul. 15, 2004, and in PCT Application No. PCT US2006031229, filed Aug. 9, 2006, the disclosures of which are incorporated by reference herein in their entirety.

Also encompassed within the invention are methods for identifying inhibitors of PAK and PIX activity and regulatory pathways which regulate leukocyte function.

The present application encompasses the use of siRNA for blocking the pathways identified herein. An siRNA of the invention can be further used with other regulators described herein, or known in the art, such as peptides, antisense oligonucleotides, nucleic acids encoding peptides described herein, aptamers, antibodies, kinase inhibitors, and drugs/agents/compounds. In another embodiment, the invention provides siRNA directed against proteins of the signal transduction pathways described herein.

In a further aspect, a first siRNA can be used in combination with a second siRNA with a slightly different sequence than the first, or the second siRNA can be directed against a different sequence altogether.

In one aspect, the invention encompasses the use of high throughput screening of siRNA and combinatorial chemical libraries.

### BRIEF DESCRIPTION OF THE DRAWINGS

FIG. 1. Flow cytometry of whole mouse blood showing uptake of fluorescently labeled PAK peptide (abscissa) in neutrophils (open histogram). Red blood cells were lysed using standard procedures, and neutrophils were identified as CD45+7-4+Gr-1⁺. PAK peptide was injected i.v. at a dose of 1 milligram (mg) at 12 hours (h) before the blood sample was taken. The grey histogram shows neutrophils from a mouse that was not injected with PAK peptide.

FIG. 2, comprising FIGS. 2A and 2B, graphically illustrates that PAK peptide-positive neutrophils cannot migrate into the BAL. Lung was harvested and digested as described (Reutershan et al., 2005, Am. J. Physiol Lung Cell Mol. Physiol. 289: L807-815), BAL was harvested, and both were subjected to flow cytometry. In mice injected with fluorescently labeled PAK peptide (abscissa), neutrophils (open histogram) show defective LPS-induced migration into the BAL (FIG. 2B). Whereas in the blood, most PMNs are PAK peptide+ (FIG. 1), only one-third of PMNs in the lung (FIG. 2A) contain PAK peptide, and even fewer in the BAL (FIG. 2B). Red blood cells were lysed using standard procedures, and neutrophils were identified as CD45⁺7-4⁺Gr-1⁺. PAK peptide was injected i.v. at a dose of 1 mg at 12 h before the blood sample was taken. The grey histogram shows neutrophils from a mouse that was not injected with PAK peptide.

FIG. 3. Number of PMNs (millions) in the vascular compartment (IV, black), the interstitial lung compartment (IS, hatched) and the BAL (BAL, white) in response to LPS. PAK peptide, but not control peptide, inhibits neutrophil migration to BAL by 70% and to IS by 60% (statistically significant, p<0.01). LPS was administered as an aerosol for 30 min. Control: no LPS. PAK control: inactive control peptide.

FIG. 4. The chemokine MIP-2 (CXCL2) induces PMN migration (left grey bar). Incubating the PEC with tat-PAK significantly reduces PMN migration (second grey bar). A similar effect is achieved when the PMNs are incubated with tat-PAK (third grey bar). The PMN and PEC effects are additive (right grey bar). Similar observations when no chemokine is present (random migration, white bars). When both PMNs and PECs are exposed to tat-PAK, migration is almost completely inhibited (statistically significant, p<0-001).

FIG. 5. PAK activity is required for CXCL1-induced cytoskeletal remodeling. Human PMNs were plated on fibronectin-coated glass slides, treated without (control) or with CXCL1 alone, with PAK or with control peptide, fixed and stained for F-actin. Photomicrographs are depicted in FIG. 5A (comprising four panels/micrographs). In FIG. 5A, the upper left panel is the control, the upper right panel is CXCL1, the lower left panel is CXCT1+ peptide, and the lower right panel is CXCL1+ control peptide. FIG. 5B graphically illustrates the F-actin content in suspended PMNs analyzed by flow cytometry.

FIG. 6, comprising FIGS. 6A, 6B, and 6C, graphically illustrates the role of PAK in adhesion and oxidative burst in human PMNs. Human PMNs were calcein-labeled and incubated in fibrinogen-coated wells for 2 hours with or without CXCL1. FIG. 6A depicts four groups. The first control group was not treated with CXCL1 (left bar), while the other three groups were treated with CXCL1 (second bar) or CXCL1 plus PAK (third bar) or CXCL1 plus control peptide (fourth bar). CXCL1 treatment alone induced a significant (P<0.05) increase in cell adhesion (second bar). The PAK peptide but not the control peptide significantly reduced adhesion (P<0.05) (FIG. 6A). Adhesion-induced superoxide production was measured as SOD-inhabitable reduction of cytochrome c. Both the inhibitory PAK peptide and dihydrocytochalasin B (dhCB) reduced oxidative activity of TNF-α-primed adherent PMNs similarly, indicating a critical role of PAK in superoxide production (6B). Respiratory burst at maximal response without (left) or with (right) TNF-α. Superoxide production by adherent PMNs treated with TNF-α was set equal to 100% (6C). *P<0.05 versus negative control, # P<0.05 versus positive control. PAK peptide had no effect on oxidative burst in cell suspension (data not shown).

FIG. 7, comprising FIGS. 7A to 7C, depicts CXCR2-dependent PAK phosphorylation and in vitro transmigration. FIG. 7A represents a western blot depicting the results of CXCL2/3 (100 ng/ml) induced PAK-phosphorylation in murine PMNs with a peak between 15 and 30 minutes (7A). Calcein-labeled PMNs from C57B1/6 mice were allowed to migrate through 3 μm Transwell filters with or without CXCL2/3 added to the lower well. Migration was reduced when PMNs were pretreated with the inhibitory PAK peptide (black bars) (7B). PMN migration across a layer of pulmonary endothelial cells was investigated using a similar protocol except that PMNs, endothelial cells, or both were pretreated with PAK peptide and washed before adding media without (open bars) or with CXCL2/3 (black bars, 250 ng/ml) to the lower wells (7C). Mean fluorescence was corrected for baseline fluorescence (endothelial cells only). Mean±SEM of n=3 experiments. *P<0.05 versus positive control.

FIG. 8, comprising FIGS. 8A, 8B, and 8C, demonstrates in vivo distribution of the PAK peptide. A FITC-tagged PAK peptide was injected intraperitoneally. Six hours later, a single cell suspension from the lungs was prepared for flow cytometry. FITC-positive cells were gated by side scatter (SSC) and FITC. FIG. 5A, comprising left (PAK) and right (CD45) panels, graphically illustrates that cells which had taken up PAK peptide were found to be 80% CD45⁺. FIG. 8B, comprising left (control) and right panels, represents images of confocal micrographs which confirmed uptake of the peptide in the lung. FIG. 8C is a graphic illustration of the fact that intravital microscopy of venules in the mouse cremaster muscle showed significant leukocyte arrest in response to CXCL1 (500 ng, injected i.v., open symbols), which was significantly reduced when PAK-activity was inhibited (black symbols). Data are mean±SEM from 4 vessels in each of n=3 mice. *P<0.05 versus control peptide.

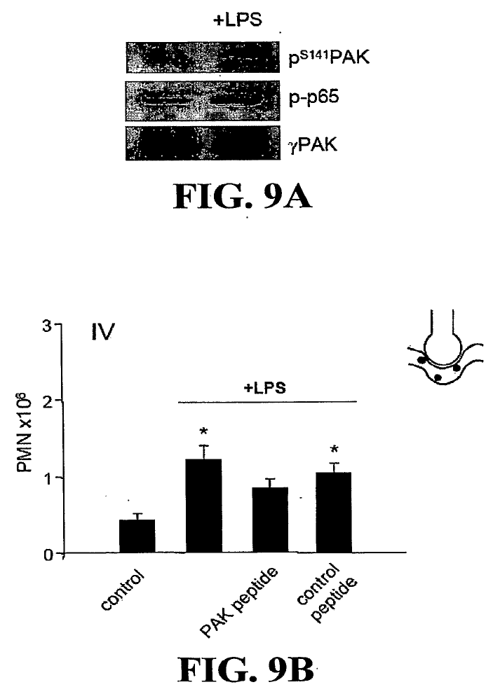

FIG. 9, comprising FIGS. 9A to 9D, demonstrates the role of PAK in LPS-induced migration of PMNs into the lung. FIG. 9A represents a western blot analysis of an experiment in which mice received an intraperitoneal injection of the inhibitory PAK peptide prior to LPS inhalation. Lung extracts were analyzed by western blotting for NF-κB p65 phosphorylation on ser536 (p-p65), for PAK phosphorylation on ser141 (p^{s141}PAK), or for total PAK2 (γPAK) as a loading control (9A). FIGS. 9B to 9C graphically illustrate accumulation of PMNs in the vasculature (IV) (9B), the lung interstitium (IS) (9C), and the bronchoalveolar space (BAL) (9D). In FIGS. 9B to 9D, the second to fourth bars are after LPS aerosol treatment. Values are means±SEM, n=4. *P<0.05. versus negative control without LPS, # P<0.05 versus positive control with LPS.

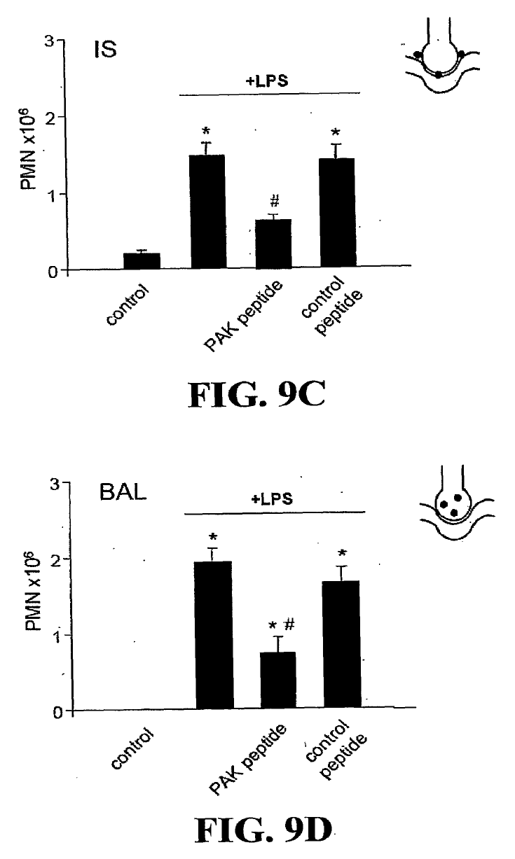

FIG. 10, comprises FIG. 10A (2 panels) and 10B (3 panels). The right panel of FIG. 10A is gated on CD45 (see left panel). FIG. 10B is gated on the upper right quadrant of FIG. 10A, right panel (neutrophils). This figure graphically illustrates PMNs (identified as CD45⁺GR1_{high} 7/4^{high}; 10A), and in blood, lung tissue, and BAL as analyzed for uptake of fluorescently labeled PAK inhibitory peptide 12 hours after LPS-inhalation (10B; open histograms). Data representative of 3 experiments. Shaded histograms indicate background fluorescence in uninjected nice.

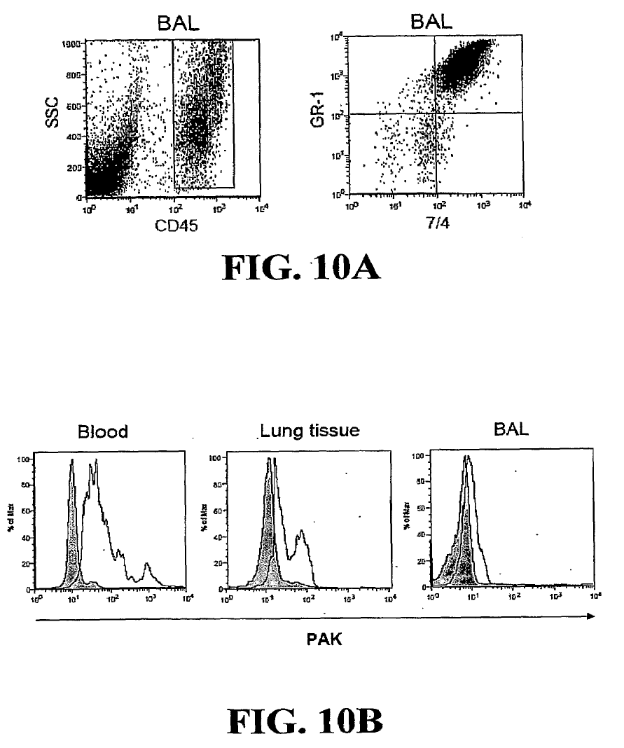

FIG. 11, comprising FIGS. 11A (four panels) and 11B (four panels), graphically illustrates the results of an analysis of lung homogenates analyzed for pPAK-expressing cells. In the resting lung (11A), the majority of all leukocytes (all Leu, CD45⁺) were pPAK-negative. Characterization of the cell types revealed that lymphocytes (CD45⁺, GR-1⁻¹) did not express pPAK. Some pPAK-positive PMNs (CD45⁺, GR-1⁻) were present in the resting lung. LPS inhalation (11B) led to a marked increase in pPAK-positive PMNs, while most lymphocytes remained pPAK-negative. Data representative of four experiments in each group.

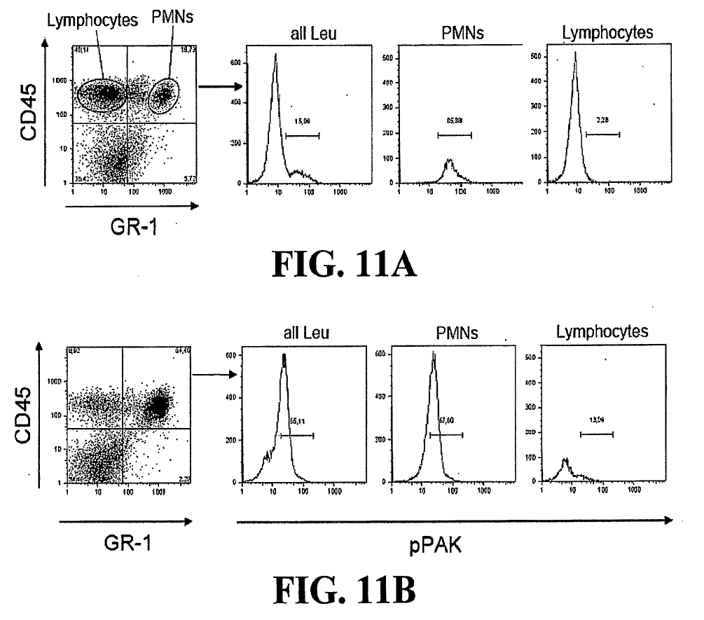

### DETAILED DESCRIPTION OF. THE INVENTION

### Abbreviations and Acronyms

ALI—acute lung injury
ANOVA—one way analysis of variance
ARDS—acute respiratory distress syndrome
BAEC—bovine aortic endothelial cells
BAL—bronchoalveolar lavage fluid
dhCB—dihydrocytochalasin B
DMEM—Dulbecco's modified Eagle's medium
ECGS—endothelial cell growth supplement
ECL—enhanced chemiluminescence
FITC—fluorescein isothiocyanate
LPS—lipopolysaccharide
PAK—p21-activated kinase
PEC— pulmonary endothelial cells
PIX—PAK-associated guanine nucleotide exchange factor
PMN—polymorphonuclear leukocytes
ROS—reactive oxygen species
SOD—superoxide dismutase
TNF—tumor necrosis factor

### DEFINITIONS

In describing and claiming the invention, the following terminology will be used in accordance with the definitions set forth below.

As used herein, the articles “a” and “an” refer to one or to more than one, i.e., to at least one, of the grammatical object of the article. By way of example, “an element” means one element or more than one element.

As used herein, the term “affected cell” refers to a cell of a subject afflicted with a disease or disorder, which affected cell has an altered phenotype relative to a subject not afflicted with a disease, condition, or disorder.

Cells or tissue are “affected” by a disease or disorder if the cells or tissue have an altered phenotype relative to the same cells or tissue in a subject not afflicted with a disease, condition, or disorder.

As used herein, “amino acids” are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

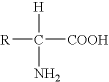

Amino acids may be classified into seven groups on the basis of the side chain R: (1) aliphatic side chains; (2) side chains containing a hydroxylic (OH) group; (3) side chains containing sulfur atoms; (4) side chains containing an acidic or amide group; (5) side chains containing a basic group; (6) side chains containing an aromatic ring; and (7) proline, an imino acid in which the side chain is fused to the amino group.

As used herein, the term “conservative amino acid substitution” is defined herein as exchanges within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

A “pathoindicative” cell is a cell which, when present in a tissue, is an indication that the animal in which the tissue is located (or from which the tissue was obtained) is afflicted with a disease or disorder.

A “pathogenic” cell is a cell which, when present in a tissue, causes or contributes to a disease or disorder in the animal in which the tissue is located (or from which the tissue was obtained).

A tissue “normally comprises” a cell if one or more of the cell are present in the tissue in an animal not afflicted with a disease or disorder.

The term “competitive sequence” refers to a peptide or a modification, fragment, derivative, or homolog thereof that competes with another peptide for its cognate binding site.

The term “complex”, as used herein in reference to proteins, refers to binding or interaction of two or more proteins. Complex formation or interaction can include such things as binding, changes in tertiary structure, and modification of one protein by another, such as phosphorylation.

A “compound,” as used herein, refers to any type of substance or agent that is commonly considered a drag, or a candidate for use as a drug, as well as combinations and mixtures of the above.

“Cytokine,” as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets and effector activities of these cytokines have been described.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a fisher decrease in the animal's state of health.

A disease, condition, or disorder is “alleviated” if the severity of a symptom of the disease or disorder, the frequency with which such a symptom is experienced by a patient, or both, are reduced.

A “fragment” or “segment” is a portion of an amino acid sequence, comprising at least one amino acid, or a portion of a nucleic acid sequence comprising at least one nucleotide. The terms “fragment” and “segment” are used interchangeably herein. A “biologically active fragment” of a peptide or protein is one which retains activity of the parent peptide such as binding to a natural ligand or performing the function of the protein.

As used herein, a “functional” biological molecule is a biological molecule in a form in which it exhibits a property or activity by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastm” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “inhibit,” as used herein, refers to the ability of a compound or any agent to reduce or impede a described function. Preferably, inhibition is by at least 10%, more preferably by at least 25%, even more preferably by at least 50%, and most preferably, the function is inhibited by at least 75%. The term “inhibit” is used interchangeably with “prevent” and “block’.

The term “inhibit a complex”, as used herein, refers to inhibiting the formation of a complex or interaction of two or more proteins, as well as inhibiting the function or activity of the complex. The term also encompasses disrupting a formed complex. However, the term does not imply that each and every one of these functions must be inhibited at the same time.

The term “inhibit a protein”, as used herein, refers to any method or technique which inhibits protein synthesis, levels, activity, or function, as well as methods of inhibiting the induction or stimulation of synthesis, levels, activity, or function of the protein of interest. The term also refers to any metabolic or regulatory pathway which can regulate the synthesis, levels, activity, or function of the protein of interest. The term includes binding with other molecules and complex formation. Therefore, the term “protein inhibitor” refers to any agent or compound, the application of which results in the inhibition of protein function or protein pathway function. However, the term does not imply that each and every one of these functions must be inhibited at the same time.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, an “instructional material” includes a publication, a recording, a diagram, or any other medium of expression which can be used to communicate the usefulness of a compound of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

The term “leukocyte”, as used herein, includes, but is not limited to, any leukocyte, including neutrophils, eosinophils, basophils, T lymphocytes, B lymphocytes, natural killer (NK) cells, NKT cells, monocytes, macrophages, dendritic cells, and the derivatives and motile precursors of these leukocytes.

The term “leukocyte-associated disease or disorder” as used herein refers to a disease or disorder where an activity or function of a leukocyte promotes or contributes to some aspect of the disease or disorder, such as inflammation. Examples include, but are not limited to, neutrophil-dependent acute lung injury.

The term “leukocyte function”, as used herein, includes functions and activities of leukocytes, including, but not limited to, polymorphonuclear (neutrophils) cells. Functions and activities encompassed within the definition include, but are not limited to, adhesion, migration, arrest, activation of PAK and activity in the cells, induction and release of ROS (oxidative burst), cytoskeletal reorganization, as well as upstream and downstream regulation of PAK and PAK activity and associated pathways.

The term “leukocyte migration”, as used herein, refers to any leukocyte movement, including transmigration, transepithelial migration, and transendothelial migration, as well as recruitment (i.e., chemotaxis) of leukocytes to sites of inflammation.

The methods and compositions encompassed by the present invention are also useful for treating chronic lung injury or diseases or conditions associated with an influx of neutrophils.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions. As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “Oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “PAK function,” as used herein, refers to any activity or function of p21-activated kinase, including, but not limited to, PAK binding with other molecules, kinase activity, autophosphorylation, translocation, activation by other molecules, etc. “PAK function” is used interchangeably with “PAK activity” herein. As used herein, “inhibition of PAK” refers to inhibiting any PAK activity or function, including inhibiting PAK synthesis.

The term “peptide” typically refers to short polypeptides.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

A “recombinant polypeptide” is one which is produced upon expression of a recombinant polynucleotide.

A peptide encompasses a sequence of 2 or more amino acids wherein the amino acids are naturally occurring or synthetic (non-naturally occurring) amino acids. Peptide mimetics include peptides having one or more of the following modifications:

1. peptides wherein one or more of the peptidyl —C(O)NR— linkages (bonds) have been replaced by a non-peptidyl linkage such as a—CH2-carbamate linkage (—CH2OC(O)NR—), a phosphonate linkage, a —CH2-sulfonamide (—CH2-S(O)2NR—) linkage, a urea (—NHC(O)NH—) linkage, a—CH2-secondary amine linkage, or with an alkylated peptidyl linkage (—C(O)NR—) wherein R is C1-C4 alkyl;

2. peptides wherein the N-terminus is derivatized to a —NRR1 group, to a —NRC(O)R group, to a —NRC(O)OR group, to a —NRS(O)2R group, to a—NHC(O)NHR group where R and R1 are hydrogen or C1-C4 alkyl with the proviso that R and R1 are not both hydrogen;

3. peptides wherein the C terminus is derivatized to —C(O)R2 where R 2 is selected from the group consisting of C1-C4 alkoxy, and —NR3R4 where R3 and R4 are independently selected from the group consisting of hydrogen and C1-C4 alkyl.

The term “permeability,” as used herein, refers to transit of fluid, cell, or debris between or through cells and tissues.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purifed” compound as used herein refers to a compound that is greater than 90% pure.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

The term “regulate” refers to either stimulating or inhibiting a function or activity of interest.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

By the term “specifically binds,” as used herein, is meant an antibody which recognizes and binds a specific protein, but does not substantially recognize or bind other molecules in a sample, or it means binding between two or more proteins as in part of a cellular regulatory process, where said proteins do not substantially recognize or bind other proteins in a sample.

The term “standard,” as used herein, refers to something used for comparison. For example, it can be a known standard agent or compound which is administered or added and used for comparing results when adding a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of diagnosis or treatment is a mammal, including a human.

The term “symptom,” as used herein, refers to any morbid phenomenon or departure from the normal in structure, function, or sensation, experienced by the patient and indicative of disease. In contrast, a sign is objective evidence of disease. For example, a bloody nose is a sign. It is evident to the patient, doctor, nurse and other observers.

As used herein, the term “treating” includes prophylaxis of the specific disease, disorder, or condition, or alleviation of the symptoms associated with a specific disease, disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

Some examples of diseases which may be treated according to the methods of the invention are discussed herein. The invention should not be construed as being limited solely to these examples, as other leukocyte-associated diseases which are at present unknown, once known, may also be treatable using the methods of the invention.

Peptides encompassed within the present invention include:

| | |
|---|---|
| SEQ ID NO:1 | YGRKKRRQRRRGKPPAPPMRNTSTM |
| SEQ ID NO:2 | KPPAPPMRNTSTM |
| SEQ ID NO:3 | YGRKKRRQRRRG |
| SEQ ID NO:4 | PPPVIAPRPEHTKSVYTR |
| SEQ ID NO:5 | YGRKKRRQRRRG PPPVIAPRPEHTKSVYTR |
| SEQ ID NO:6 | YGRKKRRQRRRGPPPVIAPAAEHAKSVYTR |

SEQ ID NO:1 consists of the sequence KPPAPPMRNTSTM (SEQ ID NO:2) from the first proline-rich domain of PAK, fused to the polybasic sequence YGRKKRRQRRRG (SEQ ID NO:3) from the HIV TAT protein (Schwarze et al, 1999, Science 285:1569) which promotes entry into cells. The peptide (SEQ ID NO:1) inhibits PAK function similarly to full length dominant negative constructs. The peptide does not block PAK kinase activity per se, but instead displaces PAK from sites of action including cell-cell junctions, which is sufficient to prevent its effects on cellular contractility, migration, and permeability. The sequence within PAK which binds to PIXα and PIXβ is PPPVIAPRPEHTKSVYTR (SEQ ID NO:4). SEQ ID NO:5 is the combination of sequences of SEQ ID NOs:3 and 4. SEQ ID NO:6 is a control peptide of SEQ ID NO:5, comprising two amino acid mutations/substitutions (indicated in bold face in SEQ ID NO:6 above).

The present invention is directed to compositions and methods for inhibiting leukocyte function. In one aspect, the leukocyte is a neutrophil. In one aspect, the function being inhibited is associated with a lung disease or disorder or acute or chronic inflammation.

In one embodiment, the invention provides a method of inhibiting leukocyte function by blocking the binding of PAK to PIX. In one aspect, the invention provides a peptide that blocks binding of PAK to PIX. The invention further encompasses analogs, homologs, derivatives, and modifications of the peptides.

The invention encompasses inhibitors, including, but not limited to, peptides, antibodies, aptamers, antisense oligonucleotides, oligonucleotides, and siRNA.

In another embodiment, the inhibitor of PAK activity or function can block other proteins or molecules from binding with PAK. In one aspect, the inhibitor of leukocyte function binds with other proteins or molecules and inhibits them from interacting with PAK. In another aspect, the inhibitor binds to PAK and inhibits other proteins or molecules from binding with PAK. In one aspect, an inhibitor of the invention inhibits the interaction of PAK with PIX. In one aspect, the inhibitor is a peptide. In one aspect, the peptide has been modified to include a sequence which aids entry of the peptide into a cell.

The present application further encompasses the use of siRNA for blocking the pathways identified herein. An siRNA of the invention can be further used with other regulators described herein, or known in the art, such as peptides, antisense oligonucleotides, nucleic acids encoding peptides described herein, aptamers, antibodies, kinase inhibitors, and drugs/agents/compounds.

Many assays and methods are described herein or are known in the art that allow one of ordinary skill in the art to monitor whether a compound regulates the components of the signal transduction and regulatory pathways of PAK and PIX, and these assays and methods are encompassed within the methods of the invention. Such assays are also useful for identifying regulators of the proteins and pathways.

For example, PAK activity and function can be monitored by assaying such things as PAK phosphorylation and translocation to cell-cell junctions. Such assays are described in Schwartz et al. (U.S. Pat. Pub. No. 2005/0233965, Published Oct. 20, 2005; the contents of which are incorporated by reference herein in their entirety). PAK-1, -2, and -3 are held in an inactive conformation via an interaction of the kinase domain with a sequence in the regulatory N terminus named the AID (Bokoch et al., Annu. Rev. Biochem., 2003, 72:743). Binding of activated Rac or Cdc42 to PAK leads to autophosphorylation of several sites that confer sustained increases in PAK kinase activity (Gatti et al., J. Biol. Chem., 1999, 274:32565; Chong et al., J. Biol. Chem., 2001, 276:17347). One of these sites, Ser¹⁴¹ in PAK2 (which corresponds to Ser¹⁴⁴ in PAK1), is within the AID and its phosphorylation contributes to activation by blocking the interaction of the AID with the kinase domain. To localize activated PAK in cells, an antibody that specifically recognizes the phosphorylated Ser¹⁴¹ site can be used.

To evaluate PAK phosphorylation in cells in response to a test compound/inhibitor, the compound can be compared to the effects of serum using confluent bovine aortic and human umbilical vein endothelial cells (BAEC and HUVEC, respectively), which can be serum-starved (0.5% serum) for 18 hours and then stimulated with 10% serum. Western blotting with anti-phospho-PAK Ser¹⁴¹ antibody can be use to assay changes in PAK phosphorylation. Fluorescence staining of similarly treated cells with a anti-phospho-PAK Ser¹⁴¹ (pPAK) can be used to indicate changes in PAK phosphorylation in response to serum, no treatment, and the test compound, by assaying whether the activated fraction of the protein localized mainly to cell-cell junctions.

The present invention further encompasses use of the yeast two-hybrid system to identify regulators of the proteins and pathways described herein. Such regulators can be drugs, compounds, peptides, nucleic acids, etc. Such regulators can include endogenous regulators.

Generally, the yeast two-hybrid assay can identify novel protein-protein interactions and compounds that alter those interactions. By using a number of different proteins as potential binding partners, it is possible to detect interactions that were previously uncharacterized. Secondly, the yeast two-hybrid assay can be used to characterize interactions already known to occur. Characterization could include determining which protein domains are responsible for the interaction, by using truncated proteins, or under what conditions interactions take place, by altering the intracellular environment. These assays can also be used to screen modulators of the interactions.

This invention encompasses methods of screening compounds to identify those compounds that act as antagonists (inhibit) of the protein interactions and pathways described herein. Screening assays for antagonist compound candidates are designed to identify compounds that bind or complex with the peptides described herein, or otherwise interfere with the interaction of the peptides with other cellular proteins.

Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized herein and in the art.

All assays for antagonists are common in that they call for contacting the compound or drug candidate with a peptide identified herein, or with a cell, under conditions and for a time sufficient to allow these two components to interact.

In binding assays, the interaction is binding and the complex formed can be isolated or detected in the reaction mixture. In a particular embodiment, one of the peptides of the complexes described herein, or the test compound or drug candidate is immobilized on a solid phase, e.g., on a microtiter plate, by covalent or non-covalent attachments. Non-covalent attachment generally is accomplished by coating the solid surface with a solution of the peptide and drying. Alternatively, an immobilized antibody, e.g., a monoclonal antibody, specific for the peptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with, but does not bind to a particular peptide identified herein, its interaction with that peptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991) Complete kits for identifying protein-protein interactions between two specific proteins using the two-hybrid technique are available. This system can also be extended to map protein domains involved in specific protein interactions as well as to pinpoint amino acid residues that are crucial for these interactions.

Compounds that interfere with the interaction of a peptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the peptide may be added to a cell along with the compound to be screened for a particular activity and the ability of the compound to inhibit the activity of interest in the presence of the peptide indicates that the compound is an antagonist to the peptide. The peptide can be labeled, such as by radioactivity.

Other assays and libraries are encompassed within the invention, such as the use of Phylomers® and reverse yeast two-hybrid assays (see Watt, 2006, Nature Biotechnology, 24:177; Watt, U.S. Pat. No. 6,994,982; Watt, U.S. Pat. Pub. No. 2005/0287580; Watt, U.S. Pat. No. 6,510,495; Barr et al., 2004, J. Biol. Chem., 279:41:43178-43189; the contents of each of these publications is hereby incorporated by reference herein in their entirety). Phylomers® are derived from sub domains of natural proteins, which makes them potentially more stable than conventional short random peptides. Phylomers® are sourced from biological genomes that are not human in origin. This feature significantly enhances the potency associated with Phylomers® against human protein targets. Phylogica's current Phylomer® library has a complexity of 50 million clones, which is comparable with the numerical complexity of random peptide or antibody Fab fragment libraries. An Interacting Peptide Library, consisting of 63 million peptides fused to the B42 activation domain, can be used to isolate peptides capable of binding to a target protein in a forward yeast two hybrid screen. The second is a Blocking Peptide Library made up of over 2 million peptides that can be used to screen for peptides capable of disrupting a specific protein interaction using the reverse two-hybrid system.

The Phylomer® library consists of protein fragments, which have been sourced from a diverse range of bacterial genomes. The libraries are highly enriched for stable subdomains (15-50 amino acids long). This technology can be integrated with high throughput screening techniques such as phage display and reverse yeast two-hybrid traps.

The present invention is directed to useful aptamers. In one embodiment, an aptamer is a compound that is selected in vitro to bind preferentially to another compound (in this case the identified proteins). In one aspect, aptamers are nucleic acids or peptides, because random sequences can be readily generated from nucleotides or amino acids (both naturally occurring or synthetically made) in large numbers but of course they need not be limited to these. In another aspect, the nucleic acid aptamers are short strands of DNA that bind protein targets. In one aspect, the aptamers are oligonucleotide aptamers. Oligonucleotide aptamers are oligonucleotides which can bind to a specific protein sequence of interest. A general method of identifying aptamers is to start with partially degenerate oligonucleotides, and then simultaneously screen the many thousands of oligonucleotides for the ability to bind to a desired protein. The bound oligonucleotide can be eluted from the protein and sequenced to identify the specific recognition sequence. Transfer of large amounts of a chemically stabilized aptamer into cells can result in specific binding to a polypeptide of interest, thereby blocking its function. [For example, see the following publications describing in vitro selection of aptamers: Klug et al., Mol. Biol. Reports 20:97-107 (1994); Wallis et al., Chem. Biol. 2:543-552 (1995); Ellington, Curr. Biol. 4:427-429 (1994); Lato et al., Chem. Biol. 2:291-303 (1995); Conrad et al., Mol. Div. 1:69-78 (1995); and Uphoff et al., Curr. Opin. Struct. Biol. 6:281-287 (1996)].

As used herein, an antagonist or blocking agent may comprise, without limitation, an antibody, an antigen binding portion thereof or a biosynthetic antibody binding site that binds a particular target protein; an antisense molecule that hybridizes in vivo to a nucleic acid encoding a target protein or a regulatory element associated therewith, or a ribozyme, siRNA, aptamer, or small molecule that binds to and/or inhibits a target protein, or that binds to and/or inhibits, reduces or otherwise modulates expression of nucleic acid encoding a target protein.

Aptamers offer advantages over other oligonucleotide-based approaches that artificially interfere with target gene function due to their ability to bind protein products of these genes with high affinity and specificity. However, RNA aptamers can be limited in their ability to target intracellular proteins since even nuclease-resistant aptamers do not efficiently enter the intracellular compartments. Moreover, attempts at expressing RNA aptamers within mammalian cells through vector-based approaches have been hampered by the presence of additional flanking sequences in expressed RNA aptamers, which may alter their functional conformation.

The idea of using single-stranded nucleic acids (DNA and RNA aptamers) to target protein molecules is based on the ability of short sequences (20 mers to 80 mers) to fold into unique 3D conformations that enable them to bind targeted proteins with high affinity and specificity. RNA aptamers have been expressed successfully inside eukaryotic cells, such as yeast and multicellular organisms, and have been shown to have inhibitory effects on their targeted proteins in the cellular environment.

The present application discloses compositions and methods for inhibiting the proteins described herein, and those not disclosed which are known in the art are encompassed within the invention. For example, various modulators/effectors are known, e.g. antibodies, biologically active nucleic acids, such as antisense molecules, siRNA, RNAi molecules, or ribozymes, aptamers, peptides or low-molecular weight organic compounds recognizing said polynucleotides or polypeptides.

Certain RNA inhibiting agents may be utilized to inhibit the expression or translation of messenger RNA (“mRNA”) that is associated with a phenotype of interest. Examples of such agents suitable for use herein include, but are not limited to, short interfering RNA (“siRNA”), ribozymes, aptamers, and antisense oligonucleotides.

In some instances, a range of 18-25 nucleotides is the most preferred size for siRNAs. siRNAs can also include short hairpin RNAs in which bolt strands of an siRNA duplex are included within a single RNA molecule. siRNA includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides. Such alterations can include the addition of non-nucleotide material, such as to the end(s) of the dsRNA or internally (at one or more nucleotides of the RNA).

In one embodiment, the RNA molecules contain a 3′ hydroxyl group.

Nucleotides in the RNA molecules of the present invention can also comprise non-standard nucleotides, including non-naturally occurring nucleotides or deoxyribonucleotides. Collectively, all such altered RNAs are referred to as analogs of RNA. siRNAs of the present invention need only be sufficiently similar to natural RNA that it has the ability to mediate RNA interference (RNAi).

The siRNAs based upon the sequences disclosed or encompassed herein are less than 100 base pairs, typically 30 bps or shorter, and are made by approaches known in the art.

Methods for designing double stranded RNA to inhibit gene expression in a target cell are known (see, e.g., U.S. Pat. No. 6,506,559; Elbashir et al. Methods 26:199-213, 2002; Chalk et al., Biochem. Biophys. Res. Comm. 319:264274, 2004; Cui et al. Computer Method and Programs in Biomedicine 75:67-73, 2004, Wang et al., Bioinformatics 20:1818-1820, 2004). For example, design of siRNAs (including hairpins) typically follow known thermodynamic rules (see, e.g., Schwarz, et al., Cell 115:199-208, 2003; Reynolds et al., Nat. Biotechnol. 22:326-30, 2004; Khvorova, et al., Cell 115:209-16, 2003). Many computer programs are available for selecting regions of a sequence that are suitable target sites. These include programs available through commercial sources such as Ambion, Dharmacon, Promega, Invitrogen, Ziagen, and GenScript as well as noncommercial sources such as EMBOSS, The Wistar Institute, Whitehead Institute, and others.

For example, design can be based on the following considerations. Typically, shorter sequences, i.e., less than about 30 nucleotides are selected. The coding region of the mRNA is usually targeted. The search for an appropriate target sequence optionally begins 50-100 nucleotides downstream of the start codon, as untranslated region binding proteins and/or translation initiation complexes may interfere with the binding of the siRNP endonuclease complex. Some algorithms, e.g., based on the work of Elbashir et al. (Elbashir et al. Methods 26:199-213, 2002) search for a selected sequence motif and select hits with approximately 50% G/C-content (30% to 70% has also worked). If no suitable sequences are found, the search is extended.

Other nucleic acids, e.g., ribozymes, antisense, can also be designed based on known principles. For example, Sfold (see, e.g., Ding, et al., Nucleic Acids Res. 32 Web Server issue, W135-W141, Ding & Lawrence, Nucl. Acids Res. 31: 7280, 7301, 2003; and Ding & Lawrence Nucl. Acids Res. 20:1034-1046, 2001) provides programs relating to designing ribozymes and antisense, as well as siRNAs.

In some embodiments, siRNAs are administered. siRNA therapy is carried out by administering to a subject an siRNA by standard vectors encoding the siRNAs of the invention and/or gene delivery systems such as by delivering the synthetic siRNA molecules. Typically, synthetic siRNA molecules are chemically stabilized to prevent nuclease degradation in vivo. Methods for preparing chemically stabilized RNA molecules are well known in the art. Typically, such molecules comprise modified backbones and nucleotides to prevent the action of ribonucleases. Other modifications are also possible, for example, cholesterol-conjugated siRNAs have shown improved pharmacological properties (see, e.g., Song et al. Nature Med. 9:347-351 (2003).

Antibodies directed against proteins, polypeptides, or peptide fragments thereof of the invention may be generated using methods that are well known in the art. For instance, U.S. patent application Ser. No. 07/481,491, which is incorporated by reference herein in its entirety, discloses methods of raising antibodies to peptides. For the production of antibodies, various host animals, including but not limited to rabbits, mice, and rats, can be immunized by injection with a polypeptide or peptide fragment thereof. To increase the immunological response, various adjuvants may be used depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For the preparation of monoclonal antibodies, any technique that provides for the production of antibody molecules by continuous cell lines in culture may be utilized. For example, the hybridoma technique originally developed by Kohler and Milstein (1975, Nature 256:495-497), the trioma technique, the human B-cell hybridoma technique (Kozbor et al., 1983, Immunology Today 4:72), and the EBV-hybridoma technique (Cole et al., 1985, in Monoclonal Antibodies and Cancer Therapy, Alan R. Liss, Inc., pp. 77-96) may be employed to produce human monoclonal antibodies. In another embodiment, monoclonal antibodies are produced in germ-free animals utilizing the technology described in international application no. PCT/US90/02545, which is incorporated by reference herein in its entirety.

In accordance with the invention, human antibodies may be used and obtained by utilizing human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, Inc., pp. 77-96). Furthermore, techniques developed for the production of “chimeric antibodies” (Morrison et al., 1984, Proc. Natl. Acad. Sci. USA. 81:6851-6855; Neuberger et al., 1984, Nature 312:604-608; Takeda et al., 1985, Nature 314:452-454) by splicing the genes from a mouse antibody molecule specific for epitopes of SLLP polypeptides together with genes from a human antibody molecule of appropriate biological activity can be employed; such antibodies are within the scope of the present invention. Once specific monoclonal antibodies have been developed, the preparation of mutants and variants thereof by conventional techniques is also available.

In one embodiment, techniques described for the production of single-chain antibodies (U.S. Pat. No. 4,946,778, incorporated by reference herein in its entirety) are adapted to produce protein-specific single-chain antibodies. In another embodiment, the techniques described for the construction of Fab expression libraries (Huse et al., 1989, Science 246:1275-1281) are utilized to allow rapid and easy identification of monoclonal Fab fragments possessing the desired specificity for specific antigens, proteins, derivatives, or analogs of the invention.

Antibody fragments which contain the idiotype of the antibody molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment; the Fab fragments which can be generated by treating the antibody molecule with papain and a reducing agent; and Fv fragments.

The generation of polyclonal antibodies is accomplished by inoculating the desired animal with the antigen and isolating antibodies which specifically bind the antigen therefrom.

Monoclonal antibodies directed against full length or peptide fragments of a protein or peptide may be prepared using any well known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

A nucleic acid encoding the monoclonal antibody obtained using the procedures described herein may be cloned and sequenced using technology which is available in the art, and is described, for example, in Wright et al. (1992, Critical Rev. in Immunol. 12(3,4):125-168) and the references cited therein. Further, the antibody of the invention may be “humanized” using the technology described in Wright et al., (supra) and in the references cited therein, and in Gu et al. (1997, Thrombosis and Hematocyst 77(4):755-759).

To generate a phage antibody library, a cDNA library is first obtained from mRNA which is isolated from cells, e.g., the hybridoma, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al. (1989, Molecular Cloning: A Laboratory Manual, Cold Spring Harbor, N.Y.).

Bacteriophage which encode the desired antibody, may be engineered such that the protein is displayed on the surface thereof in such a manner that it is available for binding to its corresponding binding protein, e.g., the antigen against which the antibody is directed. Thus, when bacteriophage which express a specific antibody are incubated in the presence of a cell which expresses the corresponding antigen, the bacteriophage will bind to the cell. Bacteriophage which do not express the antibody will not bind to the cell. Such panning techniques are well known in the art.

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH1) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al., 1991, J. Mol. Biol. 222:581-597. Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

In the production of antibodies, screening for the desired antibody can be accomplished by techniques known in the art, e.g., ELISA (enzyme-linked immunosorbent assay). Antibodies generated in accordance with the present invention may include, but are not limited to, polyclonal, monoclonal, chimeric (i.e., “humanized”), and single chain (recombinant) antibodies, Fab fragments, and fragments produced by a Fab expression library.

The peptides of the present invention may be readily prepared by standard, well-established techniques, such as solid-phase peptide synthesis (SPPS) as described by Stewart et al. in Solid Phase Peptide Synthesis, 2nd Edition, 1984, Pierce Chemical Company, Rockford, Ill.; and as described by Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the α-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and couple thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group such as formation into a carbodiimide, a symmetric acid anhydride or an “active ester” group such as hydroxybenzotriazole or pentafluorophenyl esters. Examples of solid phase peptide synthesis methods include the BOC method which utilized tert-butyloxcarbonyl as the α-amino protecting group, and the FMOC method which utilizes 9-fluorenylmethyloxcarbonyl to protect the α-amino of the amino acid residues, both methods of which are well known by those of skill in the art.

Incorporation of N- and/or C-blocking groups can also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dicholoromethane. Esterification of the suitably activated carboxyl function e.g. with DCC, can then proceed by addition of the desired alcohol, followed by deprotection and isolation of the esterified peptide product.

Incorporation of N-terminal blocking groups can be achieved while the synthesized peptide is still attached to the resin, for instance by treatment with a suitable anhydride and nitrile. To incorporate an acetyl-blocking group at the N-terminus, for instance, the resin-coupled peptide can be treated with 20% acetic anhydride in acetonitrile. The N-blocked peptide product can then be cleaved from the resin, deprotected and subsequently isolated.

To ensure that the peptide obtained from either chemical or biological synthetic techniques is the desired peptide, analysis of the peptide composition should be conducted. Such amino acid composition analysis may be conducted using high-resolution mass spectrometry to determine the molecular weight of the peptide. Alternatively, or additionally, the amino acid content of the peptide can be confirmed by hydrolyzing the peptide in aqueous acid, and separating, identifying and quantifying the components of the mixture using HPLC, or an amino acid analyzer. Protein sequenators, which sequentially degrade the peptide and identify the amino acids in order, may also be used to determine definitely the sequence of the peptide. Prior to its use, the peptide is purified to remove contaminants. In this regard, it will be appreciated that the peptide will be purified so as to meet the standards set out by the appropriate regulatory agencies. Any one of a number of a conventional purification procedures may be used to attain the required level of purity including, for example, reversed-phase high-pressure liquid chromatography (HPLC) using an alkylated silica column such as C4-, C8- or C18-silica. A gradient mobile phase of increasing organic content is generally used to achieve purification, for example, acetonitrile in an aqueous buffer, usually containing a small amount of trifluoroacetic acid. Ion-exchange chromatography can be also used to separate peptides based on their charge.

It will be appreciated, of course, that the peptides or antibodies, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof;

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C₁-C₅ branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the present invention are also contemplated, for example, inverted peptides in which all amino acids are substituted with D-amino acid forms.

Acid addition salts of the present invention are also contemplated as functional equivalents. Thus, a peptide in accordance with the present invention treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the invention.

The present invention also provides for homologs of proteins and peptides. Homologs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. To that end, depending on the size of the peptide, 10 or more conservative amino acid changes typically have no effect on peptide function.

Modifications (which do not normally alter primary sequence) include in vivo, or in-vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides or antibody fragments which have been modified using ordinary molecular biological techniques so as to improve their resistance to proteolytic degradation or to optimize solubility properties or to render them more suitable as a therapeutic agent. Homologs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

The present invention also provides nucleic acids encoding peptides, proteins, and antibodies of the invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

It is not intended that the present invention be limited by the nature of the nucleic acid employed. The target nucleic acid may be native or synthesized nucleic acid. The nucleic acid may be from a viral, bacterial, animal or plant source. The nucleic acid may be DNA or RNA and may exist in a double-stranded, single-stranded or partially double-stranded form. Furthermore, the nucleic acid may be found as part of a virus or other macromolecule. See, e.g., Fasbender et al., 1996, J. Biol. Chem. 272:6479-89 (polylysine condensation of DNA in the form of adenovirus).

Nucleic acids useful in the present invention include, by way of example and not limitation, oligonucleotides and polynucleotides such as antisense DNAs and/or RNAs; ribozymes; DNA for gene therapy; viral fragments including viral DNA and/or RNA; DNA and/or RNA chimeras; mRNA; plasmids; cosmids; genomic DNA; cDNA; gene fragments; various structural forms of DNA including single-stranded DNA, double-stranded DNA, supercoiled DNA and/or triple-helical DNA; Z-DNA; and the like. The nucleic acids may be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, OLIGONUCLEOTIDE SYNTHESIS: A PRACTICAL APPROACH (IRL Press, Oxford, England)). RNAs may be produce in high yield via in vitro transcription using plasmids such as SP65 (Promega Corporation, Madison, Wis.).

In some circumstances, as where increased nuclease stability is desired, nucleic acids having modified internucleoside linkages may be preferred. Nucleic acids containing modified internucleoside linkages may also be synthesized using reagents and methods that are well known in the art. For example, methods for synthesizing nucleic-acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH2-S—CH2), dimethylene-sulfoxide (—CH2-SO—CH2), dimethylene-sulfone (—CH2-SO2-CH2), 2′-O-alkyl, and 2′-deoxy-2′-fluoro phosphorothioate internucleoside linkages are well known in the art (see Uilmann et al., 1990, Chem. Rev. 90:543-584; Schneider et al., 1990, Tetrahedron Lett. 31:335 and references cited therein).

The nucleic acids may be purified by any suitable means, as are well known in the art. For example, the nucleic: acids can be purified by reverse phase or ion exchange HPLC, size exclusion chromatography or gel electrophoresis. Of course, the skilled artisan will recognize that the method of purification will depend in part on the size of the DNA to be purified.

The term nucleic acid also specifically includes nucleic acids composed of bases other than the five biologically occurring bases (adenine, guanine, thymine, cytosine and uracil).

The present invention is also directed to pharmaceutical compositions comprising the leukocyte function inhibitory compounds of the present invention. More particularly, such compounds can be formulated as pharmaceutical compositions using standard pharmaceutically acceptable carriers, fillers, solubilizing agents and stabilizers known to those skilled in the art.

The invention is also directed to methods of administering the compounds of the invention to a subject. In one embodiment, the invention provides a method of treating a subject with a leukocyte-associated disease, disorder, or condition by administering compounds identified using the methods of the invention description. It is preferred that a compound inhibits leukocyte function by at least 10% relative to a control where a compound is not being used to inhibit leukocyte function. It is more preferred that a compound of the invention inhibits leukocyte function by at least 25% relative to untreated controls. It is further preferred that a compound of the invention inhibits leukocyte function by at least 50% relative to untreated controls. It is even further preferred that a compound of the invention inhibits leukocyte function by at least 75% relative to untreated controls. It is also preferred that a compound of the invention inhibits leukocyte function by at least 90% relative to untreated controls. In yet another aspect, it is preferred that a compound of the invention inhibits leukocyte function by at least 95% relative to untreated controls. In one aspect of the invention, leukocyte function is inhibited due to inhibition of PAK function or activity. In one aspect, the leukocyte is a neutrophil. In one aspect, the disease or disorder is a lung disease or disorder. The terms “inhibit” and “block” are used interchangeably herein.

Pharmaceutical compositions comprising the present compounds are administered to an individual in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal means.

In accordance with one embodiment, a method of treating a leukocyte associated disease, disorder, or condition in a subject in need such treatment is provided. The method comprises administering a pharmaceutical composition comprising at least one leukocyte function inhibitory compound of the present invention to a patient in need thereof. Compounds identified by the methods of the invention which regulate leukocyte function via PAK or PIX pathways can be administered with known leukocyte inhibiting compounds or other medications as well. Preferably the compounds are administered to a human.

The invention also encompasses the use pharmaceutical compositions of an appropriate compound, homolog, fragment, analog, or derivative thereof to practice the methods of the invention, the composition comprising at least one appropriate compound, homolog, fragment, analog, or derivative thereof and a pharmaceutically-acceptable carrier.

The pharmaceutical compositions useful for practicing the invention may be administered to deliver a dose of between 1 ng/kg/day and 100 mg/kg/day.

Pharmaceutical compositions that are useful in the methods of the invention may be administered systemically in oral solid formulations, ophthalmic, suppository, aerosol, topical or other similar formulations. In addition to the appropriate compound, such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate compound according to the methods of the invention.

Compounds which are identified using any of the methods described herein may be formulated and administered to a mammal for treatment of the diseases disclosed herein are now described.

The invention encompasses the preparation and use of pharmaceutical compositions comprising a compound useful for treatment of the conditions, disorders, and diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys. Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology. A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin. Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose.

Known dispersing or wetting agents include, but are not limited to, naturally occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl para hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut-oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil in water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may also be prepared, packaged, or sold in a formulation suitable for rectal administration, vaginal administration, nasal, pulmonary, and parenteral administration. Nasal and pulmonary administration may be accomplished by means such as aerosols.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi liquid preparations such as liniments, lotions, oil in water or water in oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self propelling solvent/powder dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention. Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein. A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1/1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically, dosages of the compound of the invention which may be administered to an animal, preferably a human, range in amount from 1 μg to about 100 g per kilogram of body weight of the subject. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the subject.

The compound may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the subject, etc.

The invention also includes a kit comprising a compound of the invention and an instructional material which describes administering the composition to a cell or a tissue of a subject. In another embodiment, this kit comprises a (preferably sterile) solvent suitable for dissolving or suspending the composition of the invention prior to administering the compound to the subject. The invention also provides a kit for identifyg an inhibitor of leukocyte function as described herein, said kit comprising, for example, a sample of tissue or cells comprising a p21-activated kinase, a standard regulator of p21-activated kinase, an applicator, and an instructional material for the use thereof.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

The invention is now described with reference to the following examples. These examples are provided for the purpose of illustration only and the invention should in no way be construed as being limited to these examples, but rather should be construed to encompass any and all variations which become evident as a result of the teachings provided herein.

Methods

PAK Inhibitors Peptide

To block PAK function in vitro and in vivo, we used an inhibitory PAK peptide that selectively binds to an SH3 domain of the adaptor protein Nck and disrupts translocation of PAK to the cell membrane (63). The peptide contains the first proline rich domain of PAK linked to the transduction sequence from the HIV TAT protein to facilitate entry into cells. A peptide in which two prolines critical for SH3 binding were mutated to alanines was used as a control. To detect entry into cells, a peptide was synthesized with an N-terminal fluorescein (FITC) moiety (64).

The sequence of the tat-PAK peptide used was YGRKKRRQRRRGKPPAPPMRNTSTM. The PAK inhibitor is a short peptide that contains the sequence from PAK that exerts dominant negative activity (Kiosses et al, 2002, Circ. Res. 90:697). This peptide (YGRKKRRQRRRGKPPAPPMRNTSTM; SEQ ID NO:1) consists of the sequence KPPAPPMRNTSTM (SEQ ID NO:2) from the first proline-rich domain of PAK, fused to the polybasic sequence YGRKKRRQRRRG (SEQ ID NO:3) from the HIV TAT protein (Schwarze et al, 1999, Science 285:1569) which promotes entry into cells. The peptide (SEQ ID NO:1) inhibits PAK function similarly to full length dominant negative constructs. Other inhibitory peptides to PAK can be made and may work as well at this one or even better. Other PAK regulating peptides and their uses, as well as other PAK regulating molecules, are described in PCT Application No. PCT US2006031229, filed Aug. 9, 2006, which is incorporated herein by reference in it entirety.

F-Actin Formation

Human PMNs were purified from healthy donors (1-Step Polymorphs, Accurate Chemical and Scientific Corp., Westbury, N.Y.) and incubated with PAK- or control-peptide (20 μg/ml) for 1 hour. PMNs were then plated on fibronectin-coated glass slides and some were stimulated with CXCL1 (100 ng/ml) for 10 minutes. Cells were fixed and permeabilized, and F-actin was stained as described (65). Imaging was performed on a Zeiss LSM 510 confocal microscope and images were edited using Zeiss LSM Image Browser (version 3.5). Differential interference contrast microscopy was used to confirm the presence of cells. In separate experiments, F-actin content was measured in PMNs in suspension using flow cytometry (66).

PMN Adhesion Assay

Human PMNs were isolated as described above, pretreated with PAK- or control-peptide (20 μg/ml) for 1 hour, labeled with calcein AM, and allowed to adhere to fibrinogen-coated (2 μg/ml) bottoms of a 96-well plate in the presence of Ca++ and Mg++ for 2 hours. Some PMNs were stimulated with CXCL1 (100 ng/ml) as indicated. Non-adherent cells were washed off and fluorescence was measured in a plate-reader.

PMN Oxidative Burst

Oxidative burst of adherent PMNs was quantified by measuring the superoxide dismutase (SOD)-inhibitable reduction of cytochrome c as described (67). Briefly, PMNs (1.5×10⁶/ml in HBSS-0.1% human serum albumin) were incubated in polypropylene tubes (1 hour; 37° C.) in a shaking water bath with or without the PAK peptide (20 μg/ml). PMNs were transferred to a fibrinogen-coated 96 well tissue culture plate. Cytochrome c (1.44 mg/ml) and catalase (0.062 mg/ml) (both Sigma) were added to wells with or without TNF-alpha (10 U/ml). Optical density was measured at indicated times against matched controls with SOD at 550 nm. Some PMNs were treated with dihydrocytochalasin B (dhCB) (1 μg/ml) to disrupt cell spreading on the surface (68). These samples served as negative control.

Western Blotting

PMNs from C57B1/6 mice purified as described above were stimulated with CXCL2/3 (100 ng/ml) for the indicated times, washed with cold PBS, and lysed in modified RIPA buffer (50 mM Tris pH 7.4, 0.5% NP40, 0.5% deoxycholate, 150 mM NaCl; plus Sigma protease and phosphatase inhibitor cocktails). After 20 minutes, lysates were clarified by centrifugation at 14,000 g for 10 minutes and separated by SDS-PAGE. Proteins were transferred to PVDF membranes and blocked with 5% milk in Tris-buffered saline+0.1% Tween-20. Blots were probed overnight with either anti-PAK2 (1:1000; Santa Cruz Biotechnology) or pS141PAK (1:2000; Biosource) in 1% BSA-TBST. Blots were rinsed and probed with HRP-conjugated secondary antibodies in 1% BSA-TBST for 2 h at RT, then visualized using the enhanced chemiluminescence substrate (ECL) (Amersham).

In addition, lysates of whole lung tissue were probed for PAK2, phospho-Ser141 PAK and phospho-Ser536 p65, a marker for activation of nuclear factor-κB. Briefly, lungs were snap frozen and lysed in RIPA buffer (1% NP-40, 1% deoxycholate, 0.1% SDS, 50 mM Tris pH 7.4, 150 mM NaCl, protease and phosphatase I and II inhibitors). Lysates were cleared by centrifugation at 14,000×g for 20 min and protein concentration was determined using the DC protein assay (BioRad). 30 μg of protein from each sample were resolved using SDS-PAGE, transferred to PVDF membranes (BioRad), and probed as described above.

In Vitro Transmigration Assay

Bovine aortic endothelial cells (BAEC) were grown in low glucose Dulbecco's Modified Eagles Medium (DMEM) with 10% bovine calf serum (Atlanta Biologicals, Atlanta, Ga.), 100 μg/ml dihydrostreptomycin, and 60 U/ml penicillin (Sigma, St. Louis, Mo.) as described (69). Pulmonary endothelial cells (PEC) were isolated using a positive immunomagnetic selection with CD31 (Mec 13.3) (EasySep® Biotin Selection Kit, StemCell Technologies, Vancouver, BC, Canada) (70). PECs were cultured in DMEM (D-valine instead of L-valine, Chemikon, Phillipsburg, N.J.) with 10% of FCS, 20 mM HEPES, 1% penicillin and streptomycin (Invitrogen), and 50 μg/1 ml endothelial cell growth supplement (ECGS, Sigma). Endothelial cells were plated on fibronectin-coated filters in a Transwell system (6.5 mm diameter, 3.0 μm pore size, Corning Inc. Corning, N.J.) and grown until confluent (72 h). Medium was replaced with phenol-free DMEM with 1% FBS two hours before the experiment. Filters without endothelial cells served as negative controls.

PMNs from C57B1/6 mice were isolated from bone marrow using a three layer Percoll gradient (78, 66, and 54%) (71). PMNs, endothelial cells, or both were incubated with the PAK function-blocking peptide (20 μg/ml) for 1 hour. This peptide inhibits Nck binding to PAK and therefore blocks PAK translocation and activation of downstream events (72, 73). Controls were incubated with an inactive mutant of this peptide. For the final 15 minutes, PMNs were labeled with calcein AM (5 μM; Molecular Probes) and washed twice. Filters were moved to outer wells containing 400 μl of phenol-free DMEM with or without CXCL2/3 (MIP-2,250 ng/ml, PeproTech Inc.) (74). 2.5×105 PMNs were plated on filters with or without endothelial cells. Filters were incubated for 2 hours at 37° C. and fluorescence was measured in the bottom wells (excitation 485 nm; emission 530 nm).

In Vivo Distribution of the PAK Inhibitors Peptide

A FITC-tagged PAK peptide 75 was injected intraperitoneally to determine its distribution in vivo. Six hours after injection, PAK-positive cells were identified by flow cytometry and their expression of CD31 and CD45 was determined. In additional experiments, mice inhaled LPS after an intraperitoneal injection of the fluorescent PAK peptide. Twelve hours later, PMNs were identified in blood, lungs, and BAL by their expression of CD45, GR-1, and 7/4 and investigated for their peptide uptake. In some experiments, lungs from these mice were fixed for confocal microscopy. Controls did not receive the peptide.

Intravital Microscopy

One hour before cremaster muscle exteriorization, mice received intraperitoneal injections of either 1 mg PAK or control peptide. The cremaster muscle was prepared for intravital microscopy as previously described (76). Briefly, after intratracheal intubation and cannulation of the left carotid artery, the cremaster muscle was exteriorized and four postcapillary venules were visualized in each mouse (20-40 μm in diameter, Axioskop; Zeiss, Thomwood, N.Y.) with a saline immersion objective (SW 40/0.75 numerical aperture). A CCD camera (model VE-1000CD, Dage-MTI) was used for recording, and the number of arrested leukocytes were determined before and after administration of 500 ng CXCL1 as described (77). Arrest was defined as leukocyte adhesion longer than 30 seconds and expressed as cells per surface area, calculated from diameter and length of the vessel (S=π*d*1v). Leukocyte counts in the blood, vessel diameters, and wall shear rate were determined in both groups as described (78).

Murine Model of Acute Lung Injury

Wild type male C57B1/6 mice were obtained from Jackson Labs (Bar Harbor, Me.). All animal experiments were approved by the Animal Care and Use Committee of the University of Virginia. Mice were eight to twelve weeks of age. Up to four mice were exposed to aerosolized LPS in a custom-built cylindrical chamber (20×9 cm) connected to an air nebulizer (MicroAir, Omron Healthcare, Vernon Hills, Ill.). LPS from Salmonella enteritidis (Sigma Co., St. Louis, Mo.) was dissolved in 0.9% saline (506 μg/ml) and mice were allowed to inhale LPS for 30 minutes. As previously shown, this mimics several aspects of acute lung injury including PMN recruitment into all compartments of the lung, increase in vascular permeability (79), release of chemokines and disruption of the pulmonary architecture (80). Control mice were exposed to saline aerosol.

PMN Trafficking in the Lung

PMN recruitment into the different compartments of the lung pulmonary vasculature, interstitium, alveolar airspace) was assessed as described (81). Briefly, 24 hours after LPS exposure, intravascular PMNs were labeled by intravenous injection of Alexa 633-labeled GR-1 to murine PMN. After 5 minutes, mice were euthanized and non-adherent PMN were removed from the pulmonary vasculature by flushing 10 ml of PBS at 25 cm H20 through the spontaneously beating right ventricle. BAL was withdrawn and lungs were removed, minced, and digested in the presence of excess unlabeled anti-GR-1 to prevent possible binding of the injected antibody to extravascular PMN. A cell suspension was prepared by passing the digested lungs through a 70 μm cell strainer (BD Falcon, Bedford, Mass.). Total cells in BAL and lung were counted and percentage of PMNs determined by flow cytometry. In the BAL, PMNs were identified by their typical appearance in the forward/sideward scatter and their expression of CD45 (clone 30-F11), 7/4 (clone 7/4), and GR-1 (clone RB6-8C5). In the lung, the expression of GR-1 was used to distinguish intravascular (CD45+7/4+GR-1⁺) from interstitial (CD45+7/4+GR-1⁻) PMNs, which were not reached by the injected antibody 82.

pAK-Expressing Cells in the Lung

To determine whether neutrophils recruited to the lung in response to aerosolized LPS expressed pPAK, lungs were homogenized three hours after LPS exposure (controls received no LPS). Cells were permeabilized (Cytofix/Cytoperm, BD) and probed with fluorescently labeled (Zenon Rabbit IgG Kit, Molecular Probes) anti-phospho-Ser141. PAK antibody (Biosource). pPAK expression was analyzed in all leukocytes (CD45+), PMNs (CD45+, GR-1high) and lymphocytes (CD45+, GR-1-). Samples without anti-pPAK served to control for auto-fluorescence of the different cell types.

Statistical Analysis

Statistical analysis was performed with JMP Statistical Software (version 5.1, SAS Institute Inc., Cary, N.C.). Differences between the groups were evaluated by one way analysis of variance (ANOVA) followed by a post hoc Tukey test. Data were presented as mean±SEM and P<0.05 was considered statistically significant.

Results

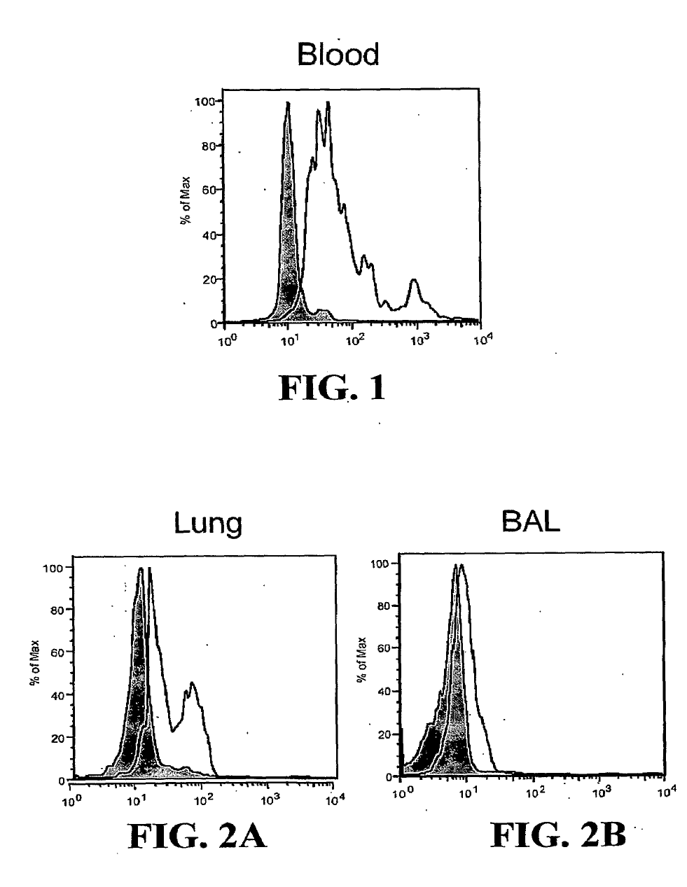

When injected intravenously, the tat-PAK peptide is taken up by neutrophils (FIG. 1). Flow cytometry of whole mouse blood demonstrated the uptake of fluorescently labeled PAK peptide into neutrophils. Red blood cells were lyzed using standard procedures, and neutrophils were identified as CD45+7-4+Gr-1⁺. PAK peptide was injected i.v. at a dose of 1 mg at 12 hours before the blood sample was taken. The grey histogram shows neutrophils from a mouse that was not injected with PAK peptide.

In an aerosolized lipopolysaccharide (LPS)-induced model of lung inflammation (Reutershan et al., 2005, Am. J. Physiol Lung Cell Mol. Physiol. 289: L807-815), PAK-peptide-positive neutrophils show reduced migration into the lung and cannot migrate into the bronchoalveolar lavage fluid (BAL), which lines the airspace.

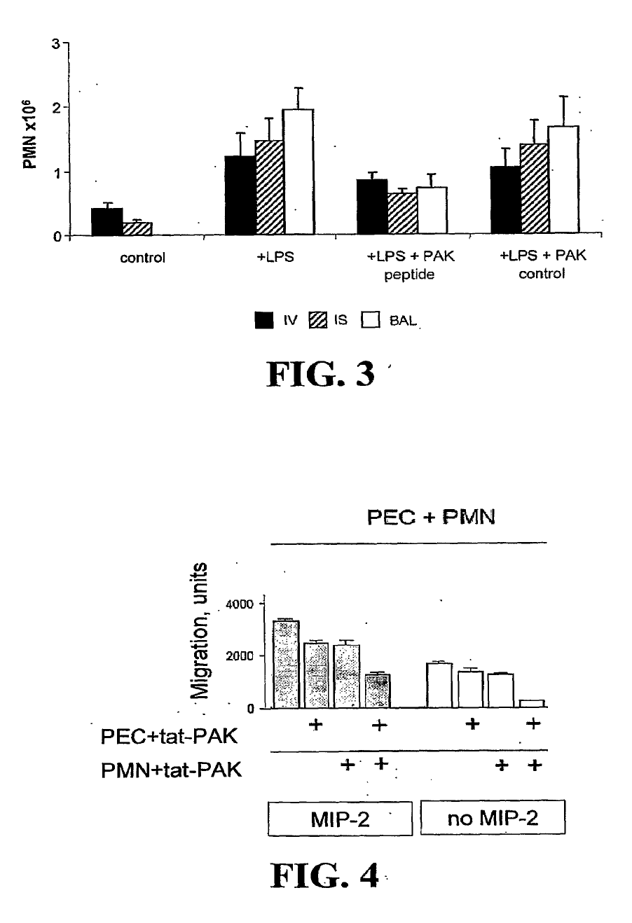

It was next demonstrated (FIG. 2) that PAK peptide-positive neutrophils cannot migrate into the BAL. Lung was harvested and digested as described (9), BAL was harvested, and both were subjected to flow cytometry. In mice injected with fluorescently labeled PAK peptide, neutrophils show defective LPS-induced migration into the BAL. Whereas in the blood, most PMNs are PAK peptide+ (FIG. 1), only one-third of PMNs in the lung contain PAK peptide, and almost all neutrophils found in the BAL are non-fluorescent, demonstrating that almost none of the PMNs that had taken up PAK peptide were able to migrate into the BAL space. Red blood cells were lysed using standard procedures, and neutrophils were identified as CD45+74+Gr-1⁺. PAK peptide was injected i-v. at a dose of 1 mg at 12 hours before the blood sample was taken. The grey histogram shows neutrophils from a mouse that was not injected with PAK peptide.

Injecting the tat-PAK peptide into mice at a dose of about 30 mg/kg inhibited PMN recruitment to the lung and the BAL in response to aerosolized lipopolysaccharide (LPS).

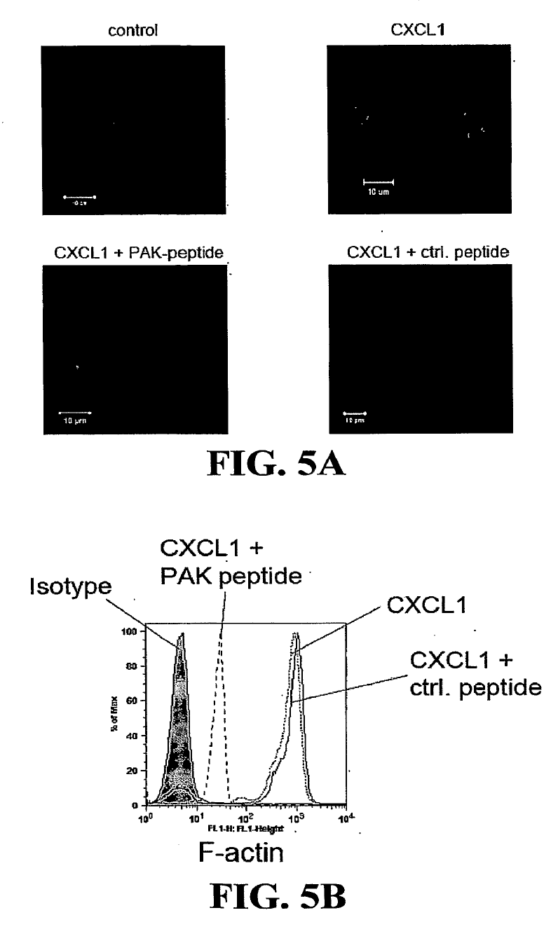

FIG. 3 demonstrates the number of PMNs (millions) in the vascular compartment, the interstitial lung compartment, and the BAL in response to LPS. The data demonstrate that PAK peptide, but not control peptide, inhibited neutrophil migration to BAL by 70% and to IS by 60% (significant, p<0.01). LPS was administered as an aerosol for 30 minutes. The control group received no LPS.

The tat-PAK peptide inhibits the permeability increase induced by PMNs co-incubated with cultured pulmonary endothelial cells (PEC) in vitro. To test whether tat-PAK had effects on PMNs separate from the known effects on endothelial cells, PEC, PMN or both were incubated with tat-PAK peptide.

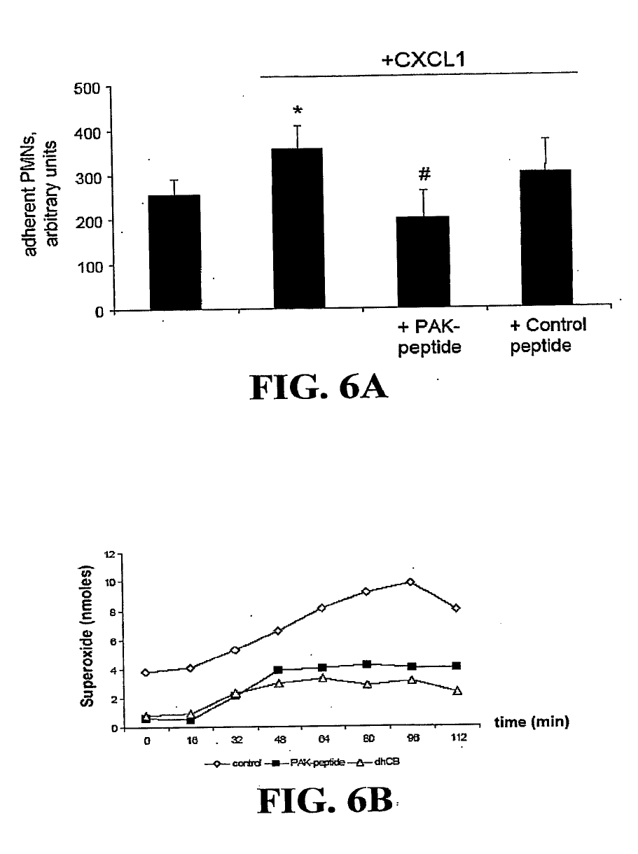

The results depicted in FIG. 4 demonstrate that the chemokine MIP-2 (CXCL2) induces PMN migration. Incubating the PEC with tat-PAK significantly reduces PMN migration. A similar effect was achieved when the PMNs were incubated with tat-PAK. The PMN and PEC effects were additive. Similar results were obtained when no chemokine were present. When both PMNs and PECs were exposed to tat-PAK, migration was almost completely inhibited (p<0.001).

PAK Regulates Cytoskeletal Reorganization in Human PMNs Remodeling of the cytoskeleton in response to an inflammatory stimulus is critical for the migratory activity of PMNs. We therefore investigated the role of PAK in actin polymerization in CXCL1-stimulated human PMNs. CXCL1-activation resulted in a marked increase in F-actin in a typical semilunar shape (FIG. 1A). Inhibition of PAK function by addition of the inhibitory peptide reduced actin polymerization substantially and prevented F-actin localization to the leading edge of the lamellipod. A control peptide in which two key prolines were mutated had no detectable effect. Quantification by flow cytometry showed that the PAK inhibitory peptide caused a ˜30-fold decrease in F-actin relative to control cells (FIG. 1B).

PMN Adhesion to Fibrinogen is PAK-Mediated

To test whether the PAK peptide impaired cell adhesion to a biological surface, we performed a static adhesion assay. Human PMNs were allowed to adhere to fibrinogen-coated wells with or without CXCL1. CXCL1 induced a significant increase of adhesion C<0.05) (FIG. 2A). The PAK inhibitory peptide reduced cell adhesion to baseline levels (P<0.05) whereas the control peptide had no effect.

Oxidative Burst in Adherent PMNs is PAK-Dependent

Unregulated release of reactive oxygen species (ROS) from PMNs can be detrimental in the setting of acute lung injury (29). ROS formation occurs upon neutrophil activation and involves integrin-dependent cytoskeletal reorganization in PMNs adherent to a surface (30, 31). We therefore investigated the role of PAK in the formation of ROS in adherent PMNs.

Oxidative burst in response to adhesion to biological surfaces was investigated as SOD-inhibitable reduction of cytochrome c. Adhesion-induced oxidative burst was markedly reduced when TNF-α-primed PMNs were pretreated with the PAK peptide (FIG. 2B+C). Similar inhibition was observed when cytoskeletal actin polymerization was disrupted by dihydrocytochalasin B (dhCB), suggesting that PAK mediates actin-dependent oxidative burst in addition to directly inducing NADPH oxidase activity (32). PAK activity did not affect the release of reactive oxygen species from PNMs in suspension (data not shown).

CXCR2-Dependent PAK Activation

CXCL2/3 (MIP-2 in mouse) is a critical CXCR2 ligand in lung injury 33, 34. To directly test whether PAK can be phosphorylated following CXCR2 ligation, murine PMNs were stimulated with CXCL2/3 (MIP-2), which induced a transient phosphorylation of PAK on ser141 in PMNs with a peak between 15 and 30 minutes (FIG. 3A), consistent with a possible role for PAK in CXCR2-dependent models of acute lung injury.

A Role for Neutrophil PAK for In Vitro Transmigration

Next, we investigated the role of PAK in PMNs for transmigration. Both baseline and CXCL2/3-stimulated migration of PMNs through a Transwell filter were reduced when PMNs were pretreated with the PAK-inhibitory peptide (P<0.05 versus untreated control) (FIG. 3B), consistent with a critical role for PAK in PNM migration. To investigate the role of PAK in transendothelial migration, pulmonary endothelial cell monolayers were grown on Transwell filters and PMN allowed to migrate to the lower well. To distinguish between effects on PMNs and endothelial cells, each cell type was pretreated with PAK inhibitory peptide for 1 h and then washed prior to beginning the assay. Migration was reduced when either PMNs or endothelial cells were pretreated with the inhibitory PAK peptide (FIG. 3C). Treatment of both populations inhibited more efficiently (P<0.05 versus untreated control). Significant inhibition was observed with both spontaneous and CXCL2/3-induced migration. Thus, PAK in both the endothelium and the PMNs contribute to transmigration.

In Vivo Distribution of the PAK Peptide

To investigate the cellular targets of the inhibitory PAK peptide in vivo, we injected mice with FITC-tagged PAK peptide. Lungs were harvested, digested, and cells positive for the PAK peptide were gated and investigated for their expression of CD31, an endothelial cell marker, and CD45, a marker for leukocytes (FIG. 4A). About 10% of PAK-positive cells were found to be CD31-positive (not shown), consistent with a previously described role of PAK in endothelial cells (35). However, the majority of PAK-positive cells (80%) were CD45 positive, suggesting a role for PAK in leukocytes. Uptake of the peptide in the lung was confirmed by confocal microscopy (FIG. 4B). The observed pattern was consistent with PAK peptide in both endothelial cells and neutrophils.

A Role for PAK in PMN Arrest

During inflammation, leukocytes roll along the endothelium and arrest after encountering chemokines. CXCR2 ligands are known to be effective arrest chemokines for PMNs (36). Chemokine-induced leukocyte arrest was investigated in the cremaster microcirculation. CXCL1 injection induced significant leukocyte arrest in the control group (FIG. 4C). When the PAK function was inhibited, leukocyte arrest was significantly reduced (P<0.05). Leukocyte counts in the blood, vessel diameters, and wall shear rate were not different between both groups (data not shown). This result implies a role for PAK in leukocyte adhesion under flow in vivo.

Pak is Required for LPS-Induced PMN Migration into the Lung

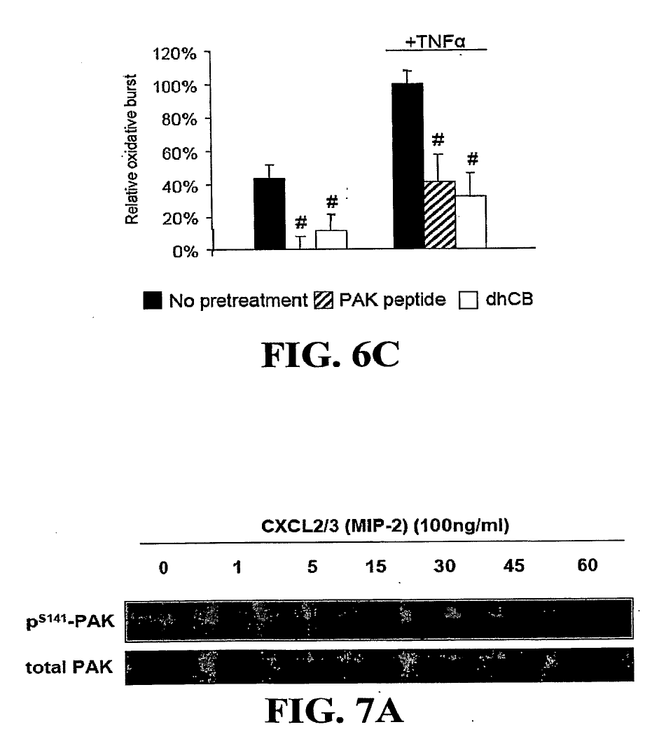

To investigate the role of PAK in a murine model of acute lung injury, mice were exposed to aerosolized LPS. This treatment induces significant PMN infiltration into the vasculature and the interstitial spaces of the lung as well as the bronchoalveolar lavage fluid (BAL) (37). LPS exposure resulted in phosphorylation of PAK2 and p65, a marker for activation of nuclear factor-κB, in total lung extracts as shown by western blot, suggesting that Pak is involved in LPS-induced lung injury (FIG. 5A). When mice were pretreated with the PAK inhibitory peptide, PMN accumulation in the vascular space was only weakly affected (FIG. 5B). However, PMN migration into the lung interstitium and alveolar space were reduced by 60 and 70%, respectively (P<0.05) (FIGS. 5C and 5D). The inactive control peptide did not affect PNM migration.

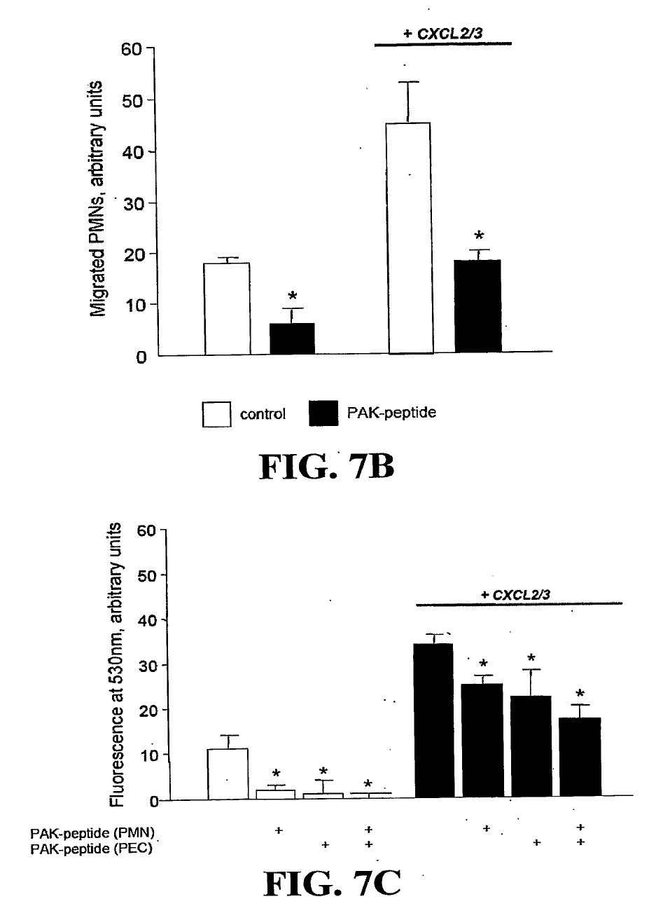

To investigate whether PMNs that had taken up PAK peptide retained their LPS-induced migratory activity into the lung, mice were injected with FITC-labeled PAK peptide and exposed to LPS. Twelve hours after LPS-inhalation, blood, lung tissue, and BAL were investigated for PMN uptake of the PAK peptide (FIG. 6). In the blood, the majority of PMNs had taken up FITC-PAK peptide. In contrast, no FITC-PAK peptide positive PMNs were found in the BAL and only some in the lung tissue. These findings suggest that uptake of the PAK peptide prevents PMN entry into the lung interstitial and alveolar space, implying a role for PAK in transendothelial (from blood to interstitium) and transepithelial (from interstitium to alveolar space) migration.

LPS Induces Recruitment of pPAK-Expressing PMNs and Macrophages

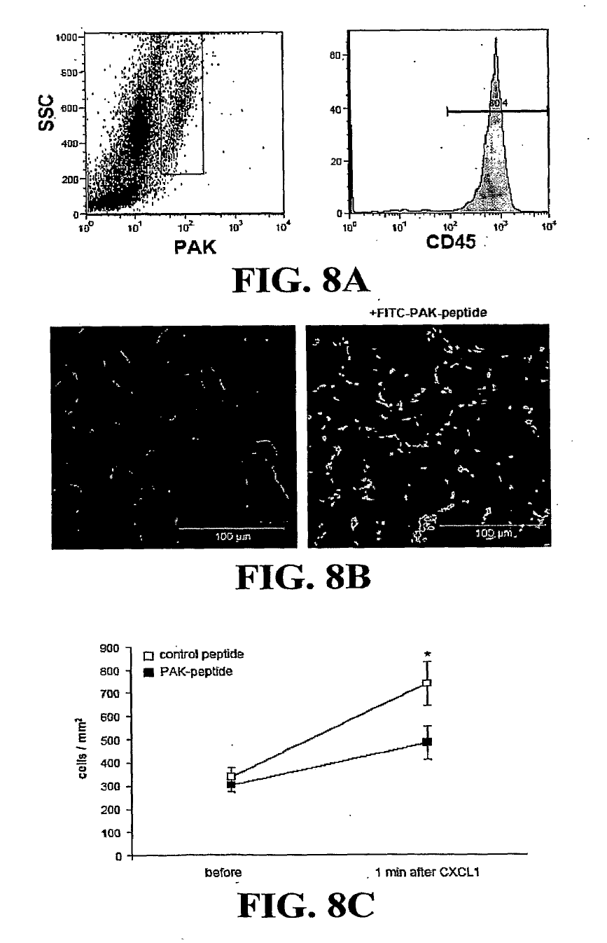

To determine whether neutrophils recruited to LPS-induced lung injury phosphorylated their PAK, we analyzed lung homogenates for their expression of pPAK. Leukocytes in resting lungs (FIG. 7A) consisted of lymphocytes (CD45⁺GR-1-), PMNs (CD45⁺GR-1^{high}), and other cells (mostly macrophages, data not shown). pPAK expression was detected in PMNs but not in lymphocytes. Three hours after LPS inhalation (FIG. 7B), the majority of leukocytes were pPAK-expressing PMNs, while lymphocytes represented a minor fraction. Most PMNs but only a minority of lymphocytes expressed pPAK.

Discussion

The present application discloses neutrophil PAK as a critical signaling molecule in LPS-induced lung inflammation. In a murine model of ALI/ARDS, inhibiting PAK substantially reduced PMN migration into lung interstitium and alveolar space. PMNs recruited to inflamed lung expressed pPAK, and PMNs that had taken up PAK inhibitory peptide could not be recruited to the inflamed lung. The present application further discloses that PAK is involved in human neutrophil activation, suggesting a potential role for PAK in regulating leukocyte-dependent inflammatory responses in inflammatory diseases.

Effects of PAK have been demonstrated in endothelial cells and other non-leukocytes (38, 39). In addition, neutrophil PAK has been implicated in mediating chemotaxis in vitro (40). PAK2 in PMNs is rapidly phosphorylated in response to fMLP 41 and other chemoattractants (42), but a role for PAK in neutrophil transmigration in vivo had not been demonstrated. Phosphorylation occurs at several sites and follows distinct kinetics in response to various stimuli (43). PAK has been implicated in NADPH oxidase-dependent superoxide release from PMNs (44) and phagocytic activity (45). PAK-dependent cytoskeletal remodeling has been demonstrated in murine PMNs where C5a failed to induce polarization of F-actin in cells lacking PIX (46). Herein are demonstrated similar results by disrupting the interaction between PAK and Nck. The inability to polarize may well be a cause for impaired neutrophil adhesion and migration in vivo. It might also explain why transmigration in vitro and in vivo was reduced in PMNs that had taken up PAK inhibitory peptide. Furthermore, it is disclosed herein that adhesion-dependent but not-independent oxidative burst of PMNs required PAK. This type of oxygen radical production is highly relevant to neutrophil-dependent tissue injury (47). The ability of PAK peptide to inhibit oxygen radical production suggests that this approach might be useful as an anti-inflammatory treatment.

Despite considerable efforts to understand PAK regulation at the molecular level and compelling evidence for the role of PAK for cell motility and migration in vitro, only a few studies have addressed its function in disease models. A recent study suggested PAK's involvement in Alzheimer's disease (57). Recent epidemiological studies indicate that the incidence of ARDS is much higher than suggested in earlier reports (58). Neutrophils are critical to the development, progression, and prognosis of the disease (59, 60) but despite advances in our understanding of the pathophysiology in ALI/ARDS, molecular mechanisms underlying neutrophil migration into the lung remain incompletely characterized (61). At this time, there are no therapeutic strategies to reduce PMN migration in ALI/ARDS. Non-specific anti-inflammatory approaches have failed to show efficacy (62). The present application utilized a model of acute lung injury because ALI and ARDS still cause significant morbidity and mortality.

The present application further discloses that neutrophils recruited to inflamed lung tissue phosphorylate their PAK. When PAK activity is inhibited by a PAK inhibitory peptide, only those neutrophils that take up the peptide fail to be recruited to the lung tissue. The PAK requirement is even more stringent for neutrophils that reach the BAL, suggesting that PAK is involved in both transendothelial and transepithelial migration. In conclusion, the present studies suggest that targeting PAK may be useful to control lung injury by reducing excessive PMN infiltration and PMN-dependent lung damage.

The disclosures of each and every patent, patent application, and publication cited herein are hereby incorporated by reference herein in their entirety.

Headings are included herein for reference and to aid in locating certain sections. These headings are not intended to limit the scope of the concepts described therein under, and these concepts may have applicability in other sections throughout the entire specification.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Accordingly, the present invention is not intended to be limited to the embodiments shown herein but is to be accorded the widest scope consistent with the principles and novel features disclosed herein.

### BIBLIOGRAPHY

1. Abraham et al., Am. J. Physiol Lung Cell Mol. Physiol. 279, L1137-L1145 (2000).
2. Azoulay et al., Crit. Care Med. 30, 781-786 (2002).
3. Ware et al., N. Engl. J. Med. 342, 1334-1349 (2000).
4. Doerschuk, J. Appl. Physiol 74, 3040-3045 (1993).
5. Worthen, Science 245, 183-186 (1989).
6. Reutershan, J. Clin. Invest. 116, 695-702 (2006).
7. Basit, Am. J. Physiol Lung Cell Mol. Physiol (2006).
8. Glogauer, Journal of Immunology 170, 5652-5657 (2003).
9. Srinivasan, J. Cell Biol. 160, 375-385 (2003).
10. Bokoch, Annu. Rev. Biochem. 72, 743-781 (2003).
11. Ding et al., J. Biol. Chem. 271, 24869-24873 (1996).
12. Wang et al., Biochemistry 41, 7100-7107 (2002).
13. Zhan, J. Immunol. 171, 3785-3793 (2003).
14. Huang et al., Mol. Cell. Biol. 18, 7130-7138 (1998).
15. Parrini, et al., Mol. Cell. 9, 73-83 (2002).
16. Lei, M. et al., Cell 102, 387-397 (2000).
17. Bokoch et al., J. Biol. Chem. 271, 25746-25749 (1996).
18. Manser et al., Mol. Cell 1, 183-192 (1998).
19. Sells et al., Curr. Biol. 7, 202-210 (1997).
20. Kiosses, Circ Res 90, 697-702 (2002).
21. Stockton et al., J. Biol. Chem. 279, 46621-46630 (2004).
22. Manser, Mol. Cell. Biol. 17, 1129-1143 (1997).
23. Sells, J. Cell Biol. 151, 1449-1458 (2000).
24. Zhao et al., Mol. Cell. Biol. 18, 2153-2163 (1998).
25. Knaus et al., Science 269, 221-223 (1995).
26. Martyn et al., Blood 106, 3962-3969 (2005).
27. Li et al., Cell 114, 215-227 (2003).
28. Kiosses et al., Circ Res 90, 697-702 (2002).
29. Moraes et al., Curr. Opin. Hematol. 13, 21-27 (2006).
30. Wang et al., Am. J. Respir. Crit. Care Med. 166, 872-877 (2002).
31. Nathan, J. Clin. Invest 80, 1550-1560 (1987).
32. Martyn et al., Blood 106, 3962-3969 (2005).
33. Sue et al., J. Immunol. 172, 3860-3868 (2004).
34. Reutershan et al., J. Clin. Invest. 116, 695-702 (2006).
35. Stockton et al., J. Biol. Chem. 279, 46621-46630 (2004).
36. Smith et al., J. Exp. Med. 200, 935-939 (2004).
37. Reutershan et al., Am J Physiol Lung Cell Mol Physiol 289, L807-L815 (2005).
38. Knaus et al., Science 269, 221-223 (1995).
39. Ding et al., J. Biol. Chem. 271, 24869-24873 (1996).
40. Li et al., Cell 114, 215-227 (2003).
41. Zhan et al., J. Immunol. 171, 3785-3793 (2003).
42. Huang et al., Mol. Cell. Biol. 18, 7130-7138 (1998).
43. Zhan et al., J. Immunol. 171, 3785-3793 (2003).
44. Martyn et al., Blood 106, 3962-3969 (2005).
45. Dharmawardhan, J. Leukoc. Biol. 66, 521-527 (1999).
46. Li et al., Cell 114, 215-227 (2003).
47. Usatyuk et al., Am. J. Physiol Lung Cell Mol. Physiol. 289, L999-1010 (2005).
48. Stockton et al., J. Biol. Chem. 279, 46621-46630 (2004).
49. Zhan et al., J. Immunol. 171, 3785-3793 (2003).
50. Wang et al., Biochemistry 41, 7100-7107 (2002).
51. Huang et al., Mol. Cell. Bibl. 18, 7130-7138 (1998).
52. Reutershan et al., J. Clin. Invest. 116, 695-702 (2006).
53. Krupa et al., Am. J. Physiol Lung Cell Mol. Physiol (2004).
54. Reutershan et al., 3. Clin. Invest. 116, 695-702 (2006).
55. Belperio et al., J. Clin. Invest 110, 1703-1716 (2002).
56. Kaneider et al., Nat. Med. 11, 661-665 (2005).
57. Zhao et al., Nat. Neurosci. 9, 234-242 (2006).
58. Rubenfeld et al., N. E Engl. J. Med. 353, 1685-1693 (2005).
59. Azoulay et al., Crit. Care Med. 30, 781-786 (2002).
60. Kurdowska et al., Crit. Care Med. 30, 2335-2337 (2002).
61. Matthay et al., Am. J. Respir. Crit. Care Med. 167, 1027-1035 (2003).
62. Adhikari et al., Treat. Respir. Med. 3, 307-328 (2004).
63. Kiosses et al., Circ. Res. 90, 697-702 (2002).
64. Kiosses et al., Circ Res 90, 697-702 (2002).
65. Reutershan et al., J. Clin. Invest. 116, 695-702 (2006).
66. Walzog et al., J. Leukoc. Biol. 56, 625-635 (1994).
67. Sullivan et al., Int. J. Immunopharmacol. 17, 793-803 (1995).
68. Fuortes et al., J. Cell Biol. 120, 777-784 (1993).
69. Stockton et al., J. Biol. Chem. 279, 46621-46630 (2004).
70. Reutershan et al., J. Clin. Invest. 116, 695-702 (2006).
71. Reutershan et al., J. Clin. Invest. 116, 695-702 (2006).
72. Kiosses et al., Circ Res 90, 697-702 (2002).
73. Zhou et al., Mol. Cell. Biol. 23, 8058-8069 (2003).
74. Reutershan et al., 3. Clin. Invest. 116, 695-702 (2006).
75. Kiosses et al., Circ Res 90, 697-702 (2002).
76. Ley et al., J. Exp. Med. 181, 669-675 (1995).
77. Smith et al., J. Exp. Med. 200, 935-939 (2004).
78. Sperandio et al., J. Exp. Med. 197, 1355-1363 (2003).
79. Reutershan et al., Am J Physiol Lung Cell Mol Physiol 289, L807-L815 (2005).
80. Reutershan et al., Anesthesiology 104, 511-517 (2006).
81. Reutershan et al., Am J Physiol Lung Cell Mol Physiol 289, L807-L815 (2005).
82. Reutershan et al., Am J Physiol Lung Cell Mol Physiol 289, L807-L815 (2005).

What is claimed is:

1. A method of inhibiting leukocyte function, said method comprising contacting said leukocyte with an effective amount of at least one inhibitor of a protein regulatory pathway selected from the group consisting of PAK and PIX regulatory pathways, thereby inhibiting leukocyte function.

2. The method of claim 1, wherein said leukocyte is a mammalian leukocyte.

3. The method of claim 2, wherein mammalian leukocyte is a human leukocyte.

4. The method of claim 3, wherein said leukocyte is selected from the group consisting of neutrophils, eosinophils, basophils, T lymphocytes, B lymphocytes, natural killer cells, NKT cells, monocytes, macrophages, dendritic cells, and derivatives and motile precursors thereof.

5. The method of claim 4, wherein said leukocyte is a neutrophil.

6. The method of claim 5, wherein said function is selected from the group consisting of adhesion, migration, arrest, activation of PAK, activation of PIX, PAK activity, PIX activity, induction of ROS formation, release of ROS, and cytoskeletal reorganization.

7. The method of claim 5, wherein said at least one inhibitor inhibits growth factor-stimulated or cytokine-stimulated neutrophil function.

8. The method of claim 7, wherein said at least one inhibitor inhibits CXCR2-dependent PAK activation.

9. The method of claim 5, wherein said at least one inhibitor inhibits bacterial toxin-stimulated neutrophil function.

10. The method of claim 5, wherein said at least one inhibitor is selected from the group consisting of peptide, nucleic acid, antisense oligonucleotide, siRNA, aptamer, kinase inhibitor, and antibody.

11. The method of claim 10, wherein said peptide comprises an amino acid sequence selected from the group consisting of SEQ ID NOs:1 and 5, and biologically active fragments, homologs, derivatives, and modifications thereof.

12. The method of claim 10, wherein said at least one inhibitor inhibits protein interaction or complex formation.

13. A method of treating a disease or disorder associated with increased leukocyte infiltration or activity in a subject in need thereof, said method comprising administering to said subject a pharmaceutical composition comprising an effective amount of at least one inhibitor of a protein regulatory pathway selected from the group consisting of PAK and PIX regulatory pathways, thereby treating a disease or disorder associated with increased leukocyte infiltration or activity.

14. The method of claim 13, wherein said disease or disorder comprises acute or chronic inflammation.

15. The method of claim 14, wherein said wherein said disease or disorder comprising acute or chronic inflammation is selected from the group consisting of arthritis, asthma, multiple sclerosis, inflammatory diseases of the central nervous system, neuritis, inflammatory diseases of the peripheral nervous system, atopic diseases, inflammatory bowel diseases, inflammatory diseases of the skin, inflammatory diseases of the mucosal membranes, hepatitis, inflammatory diseases of the kidney, sepsis, septic shock, and cancers.

16. The method of claim 13, wherein said disease or disorder is a lung disease or disorder.

17. The method of claim 16, wherein said lung disease or disorder is selected from the group consisting of acute lung injury, acute respiratory distress syndrome, ventilator lung, sepsis-induced lung failure, and pneumonia.

18. The method of claim 16, wherein said leukocyte is a neutrophil.

19. The method of claim 18, wherein said neutrophil comprises said pathway.

20. A kit for administering a compound which inhibits a disease or disorder associated with increased leukocyte infiltration or activity, said kit comprising a pharmaceutical composition comprising at least one inhibitor of a protein regulatory pathway selected from the group consisting of PAK and PIX regulatory pathways, an applicator, and an instructional material for the use thereof.

21. A method for identifying a compound that inhibits PAK or PIX regulated function of leukocytes, wherein said compound inhibits at least one protein regulatory pathway selected from the group consisting of PAK and PIX, said method comprising:
contacting a test leukocyte comprising at least one of said regulatory pathways with a test compound;
measuring the activity or function of at least one of said regulatory pathways,
wherein a lower level of said activity or function in the test leukocyte, compared with the level of activity or function in an otherwise identical leukocyte not contacted with said test compound, is an indication that said test compound inhibits leukocyte function.

22. The method of claim 21, wherein said function is selected from the group consisting of adhesion, migration, arrest, activation of PAK, activation of PIX, PAK activity, PIX activity, induction of ROS formation, release of ROS, and cytoskeletal reorganization.

23. The method of claim 21, wherein said regulatory pathway is PAK.

24. The method of claim 23, wherein said method measures PAK phosphorylation.

25. The method of claim 20, wherein said leukocyte is a neutrophil.

26. The method of claim 20, wherein said method measures protein-protein interaction or complex formation.
